(19) United States
(12) Patent Application Publication
(10) Pub. No.: US 2010/0303848 A1
(43) Pub. Date: Dec. 2, 2010

(54) VACCINATION METHOD

(75) Inventors: Barton F. Haynes, Durham, NC (US); Hua-Xin Liao, Durham, NC (US)

Correspondence Address: NIXON & VANDERHYE, PC, 901 NORTH GLEBE ROAD, 11TH FLOOR, ARLINGTON, VA, 22203, US

(73) Assignee: DUKE UNIVERSITY, Durham, NC (US)

(21) Appl. No.: 12/086,350

(22) PCT Filed: Dec. 13, 2006
(86) PCT No.: PCT/US2006/047344

§ 371 Date: Jul. 20, 2010

Related U.S. Application Data
(60) Provisional application No. 60/749,596, filed on Dec. 13, 2005.

(51) Int. Cl. A61K 39/21; C07K 14/155; C07H 21/04; C12N 15/63; A61P 31/18

(52) U.S. Cl. 424/188.1; 530/350; 530/324; 536/23.72; 435/320.1

(57) ABSTRACT

The present invention relates, in general, to human immunodeficiency virus (HIV) and, in particular, to methods of effecting local and systemic immunization against HIV while protecting cells (e.g., mucosal dendritic and epithelial cells) from viral challenge. The invention further relates to compounds (e.g., nucleic acids encoding polypeptides that can elicit an immune response and/or protect cells against viral challenge) and compositions suitable for use in such methods.

This application claims priority from U.S. Provisional Application No. 60/749,596, filed Dec. 13, 2005, the entire content of which is incorporated herein by reference.

### TECHNICAL FIELD

The present invention relates, in general, to human immunodeficiency virus (HIV) and, in particular, to methods of effecting local and systemic immunization against HIV while protecting cells (e.g., mucosal dendritic and epithelial cells) from viral challenge. The invention further relates to compounds (e.g., nucleic acids encoding polypeptides that can elicit an immune response and/or protect cells against viral challenge) and compositions suitable for use in such methods.

### BACKGROUND

The first antibodies that are made in acute HIV-1 infection are against the CD4 binding site (Moore et al, J. Virol. 68(8) 5142 (1994)), the CCR5 co-receptor binding site (Choe et al, Cell 114(2):161-170 (2003)), and the V3 loop (Moore et al, J. Acquir. Immun. Def. Syn. 7(4):332 (1994)). However, these antibodies do not control HIV-1 and are easily escaped (Burton et al, Nature Immun. 5:233-236 (2004), Wei et al, Nature 422(6929):307-312 (2003)). Neutralizing antibodies against autologous virus develop fifty to sixty days after infection, but antibodies capable of neutralizing heterologous HIV-1 strains do not arise until after the first year of infection (Richman et al, Proc. Natl. Acad. Sci. USA 100(7):4144-4149 (2003), Wei et al, Nature 422(6929):307-312 (2003)).

Egelhofer et al (J. Virol. 78:568 (2004)) have developed a gene therapy approach to inhibiting entry into cells of a broad spectrum of HIV-1 variants. The method involves introduction into cells of a retroviral vector expressing a membrane-anchored fusion inhibitory peptide derived from the C-terminal heptad repeat of the HIV-1 gp41 transmembrane glycoprotein. Entry into cells expressing the gp41-derived peptide is inhibited at the level of membrane fusion.

The present invention provides a method of effecting local and systemic immunization against HIV while protecting mucosal cells (e.g., rectal or vaginal mucosal cells), or other cells, from viral challenge during the time required for development of an immune response. The invention also provides nucleic acids, and vectors comprising same, that encode polypeptides that can elicit an immune response and/or protect viral-challenged cells.

### SUMMARY OF THE INVENTION

The invention relates to methods of effecting local and systemic immunization against HIV while protecting certain cell types, for example, vaginal and rectal cells, from viral challenge. In accordance with the invention, cell protection can be effected using a gene therapy approach. The invention further relates to compounds and compositions suitable for use in such methods.

Objects and advantages of the present invention will be clear from the description that follows.

### BRIEF DESCRIPTION OF THE DRAWINGS

FIGS. 1A-1E: To utilize gene (nucleic acid) inserts including those listed below as HIV-1 vaccine immunogens, these genes (nucleic acids) can be codon optimized, cloned into plasmids for generating DNA vaccines, recombinant adenoviruses, recombinant mycobacteria and recombinant vaccinia viruses and as well as for production of recombinant proteins. For expressing these inserts, except the full-length gp160 constructs, a leader sequence can be included:

“TCTAGAGCCGCCATGCGCGTGCGCGGCATCCAGCGCAACTGCCAGCACCTGTGGCG CTGGGGCACCCTGATCCTGGGCATGCTGATGATCTGCTCCGCCGCC” , derived from the sequence that encodes for the N-terminus of CON-S Env (MRVRGIQRNCQHLWRWGTLILGMLMICSAA), as protein synthesis initiation and maturation signal. FIG. 1A. CON-S env01 gp41. FIG. 1B. A.con.env03 gp41. FIG. 1C. B.con.env03 gp41. FIG. 1D. C.con.env03 gp41. FIG. 1E.JRFL.env gp41.

FIGS. 2A-2E. FIG. 2A. CON-S env01 gp41ΔF. FIG. 2B. A.con.env03 gp41ΔF. FIG. 2C. B.con.env03 gp41ΔF. FIG. 2D. C.con.env03 gp41ΔF. FIG. 2E. JRFL.env gp41ΔF.

FIGS. 3A-3E. FIG. 3A—CON-S env01 gp41ΔFHR-1. FIG. 3B. A.con.env03 gp41ΔFHR-1. FIG. 3C. B.con.env03 gp41ΔFHR-1. FIG. 3D. C.con.env03 gp41ΔFHR-1. FIG. 3E. JRFL.env gp41ΔFHR-1.

FIGS. 4A-4E. FIG. 4A. CON-S env01 HR-2TMCyt. FIG. 4B. A.con.env03 HR-2TMCyt. FIG. 4C. B.con.env03 HR-2TMCyt. FIG. 4D C.con.env03 HR-2TMCyt. FIG. 4E. JRFL.env HR-2TMCyt.

FIGS. 5A-5E. FIG. 5A. CON-S env01 gp41ΔFHR-2ID. FIG. 5B. A.con.env03 gp41ΔFHR-2ID. FIG. 5C. B.con.env03 gp41ΔFHR-2ID. FIG. 5D C.con.env03 gp41ΔFHR-2ID. FIG. 5E. JRFL.env gp41ΔFHR-2ID.

FIGS. 6A-6E. FIG. 6A. CON-S env01 gp41ΔFCyt. FIG. 6B. A.con.env03 gp41ΔFCyt. FIG. 6C. B.con.env03 gp41ΔFCyt. FIG. 6D. C.con.env03 gp41ΔFCyt. FIG. 6E—JRFL.env gp41ΔFCyt.

FIG. 7A-7E. FIG. 7A. CON-S env01 gp41ΔFCyt. FIG. 7B. A.con.env03 gp41ΔFCyt. FIG. 7C. B.con.env03 gp41ΔFCyt. FIG. 7D. C.con.env03 gp41ΔFCyt. FIG. 7E-—JRFL.env gp41ΔFCyt.

FIGS. 8A-8E. FIG. 8A. CON-S env01 HR-2TM. FIG. 8B. A.con.env03 HR-2TM. FIG. 8C. B.con.env03 HR-2TM. FIG. 8D C.con.env03 HR-2TM. FIG. 8E. JRFL.env HR-2TM.

FIGS. 9A-9E. FIG. 9A. CON-S env01 HR-1TM. FIG. 9B. A.con.env03 HR-1TM. FIG. 9C. B.con.env03 HR-1TM. FIG. 9D C.con.env03 HR-1TM. FIG. 9E. JRFL.env HR-1TM.

FIGS. 10A-10E. FIG. 10A. CON-S env01 gp41ΔFHR-2Cyt. FIG. 10B. A.con.env03 gpΔ41FHR-2Cyt. FIG. 10C. B.con.env03 gp41ΔFHR-2Cyt. FIG. 10D. C.con.env03 gp41ΔFHR-2Cyt. FIG. 10E. JRFL.env gp41ΔFHR-2Cyt.

FIGS. 11A-11E. FIG. 11A. CON-S env01 gp41ΔTMCyt. FIG. 11B. A.con.env03 gp41ΔTMCyt. FIG. 11C. B.con.env03 gp41ΔTMCyt: FIG. 11D C.con.env03 gp41ΔTMCyt. FIG. 11E. JRFL.env gp41ΔTMCyt.

FIGS. 12A-12E. FIG. 12A. CON-S env01 gp41ΔFTMCyt. FIG. 12B. A.con.env03 gp41ΔFTMCyt. FIG. 12C. B.con.env03 gp41ΔFTMCyt. FIG. 12D C.con.env03 gp41ΔFTMCyt. FIG. 12E. JRFL.env gp41ΔFTMCyt.

FIGS. 13A-13E. FIG. 13A. CON-S env01 gp41ΔFHR-1TMCyt. FIG. 13B. A.con.env03 gp41ΔFHR-1TMCyt. FIG. 13C. B.con.env03 gp41ΔFHR-1TMCyt. FIG. 13D C.con.env03 gp41ΔFHR-1TMCyt. FIG. 13E. JRFL.env gp41ΔFHR-1TMCyt.

FIGS. 14A-14E. FIG. 14A. CON-S env01 HR-2. FIG. 14B. A.con.env03 HR-2. FIG. 14C. B.con.env03 HR-2. FIG. 14D C.con.env03 HR-2. FIG. 14E. JRFL.env HR-2.

FIGS. 15A-15E. FIG. 15A. CON-S env01 HR-1. FIG. 15B. A.con.env03 HR-1. FIG. 15C. B.con.env03 HR-1. FIG. 15D C.con.env03 HR-1. FIG. 15E. JRFL.env HR-1.

FIGS. 16A-16E. FIG. 16A. CON-S env01 p41ΔFTMCy. FIG. 16B. A.con.env03 p41ΔFTMCy. FIG. 16C. B.con.env03 p41ΔFTMCy. FIG. 16D C.con.env03 p41ΔFTMCy. FIG. 16E. JRFL.env p41ΔFTMCy.

FIGS. 17A-17E. FIG. 17A. CON-S env01 gp41ΔFHR-2TMCyt. FIG. 17B. A.con.env03 gp41ΔFHR-2TMCyt. FIG. 17C. B.con.env03 gp41ΔFHR-2TMCyt. FIG. 17D C.con.env03 gp41ΔFHR-2TMCyt. FIG. 17E. JRFL.env gp41ΔFHR-2TMCyt.

FIGS. 18A-18K. FIG. 18A. CON-S env160. FIG. 18B. CON-T Env 160. FIG. 18C. A.con.Env03. FIG. 18D B.con.Env03. FIG. 18E. C.con.Env03. FIG. 18F. JRFL Env 160. FIG. 18G 00KE-MSA4076-A (Subtype A). FIG. 18H. QH0515.1g gp160 (Subtype B). FIG. 18I. DU123.6 gp160 (Subtype C). FIG. 18J. 97CNGX2F_AE (Subtype AE_{—}01). FIG. 18K. DRCBL-F (Subtype G).

### DETAILED DESCRIPTION OF THE INVENTION

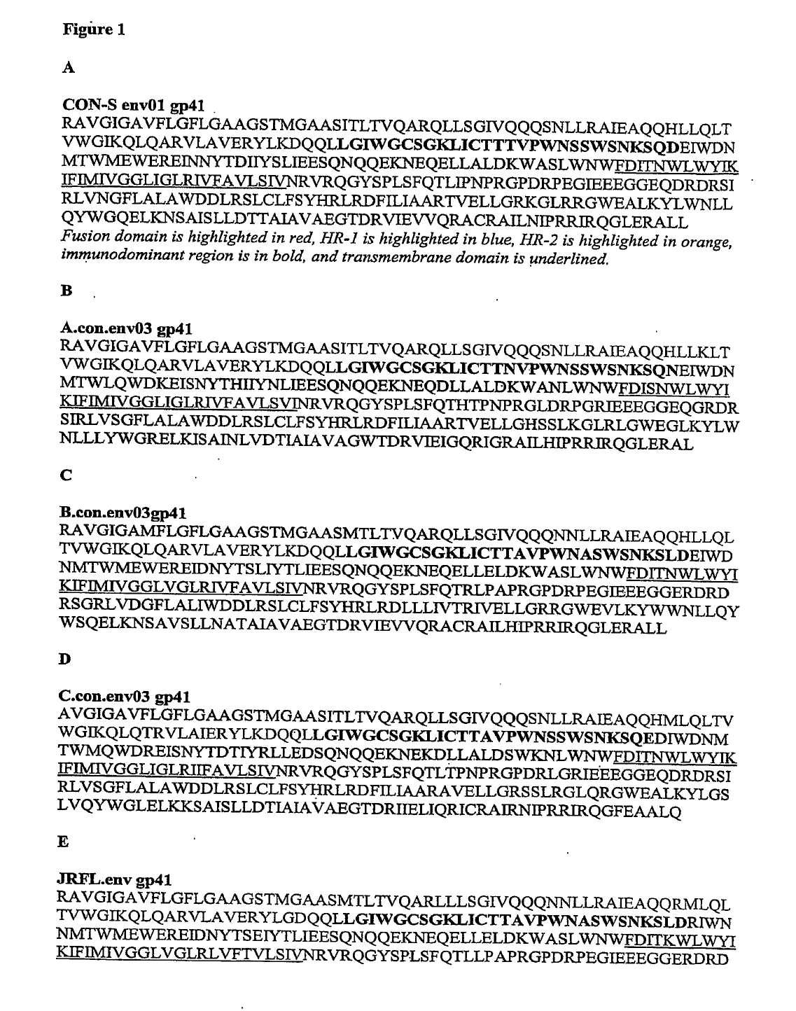

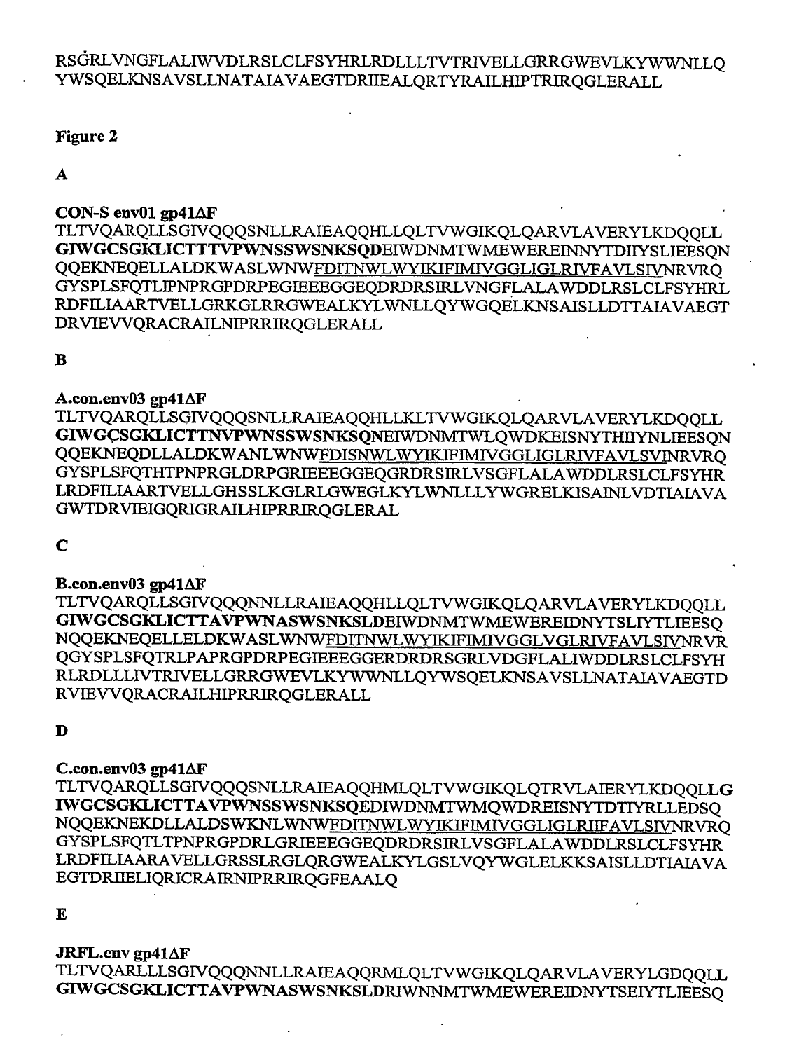

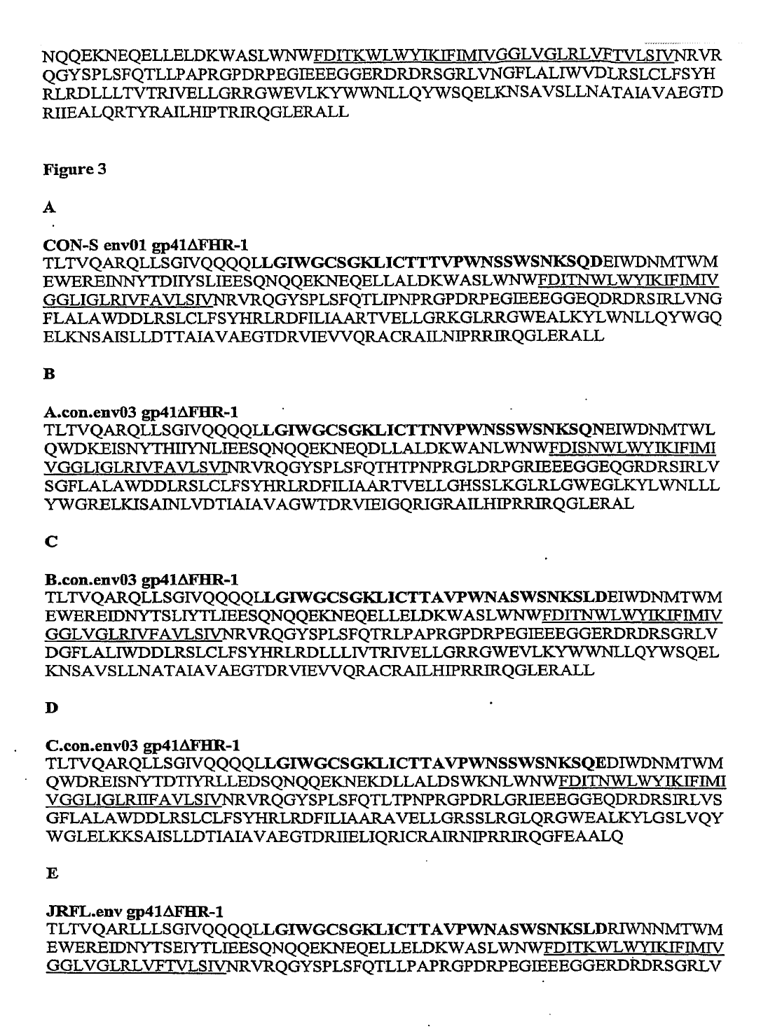

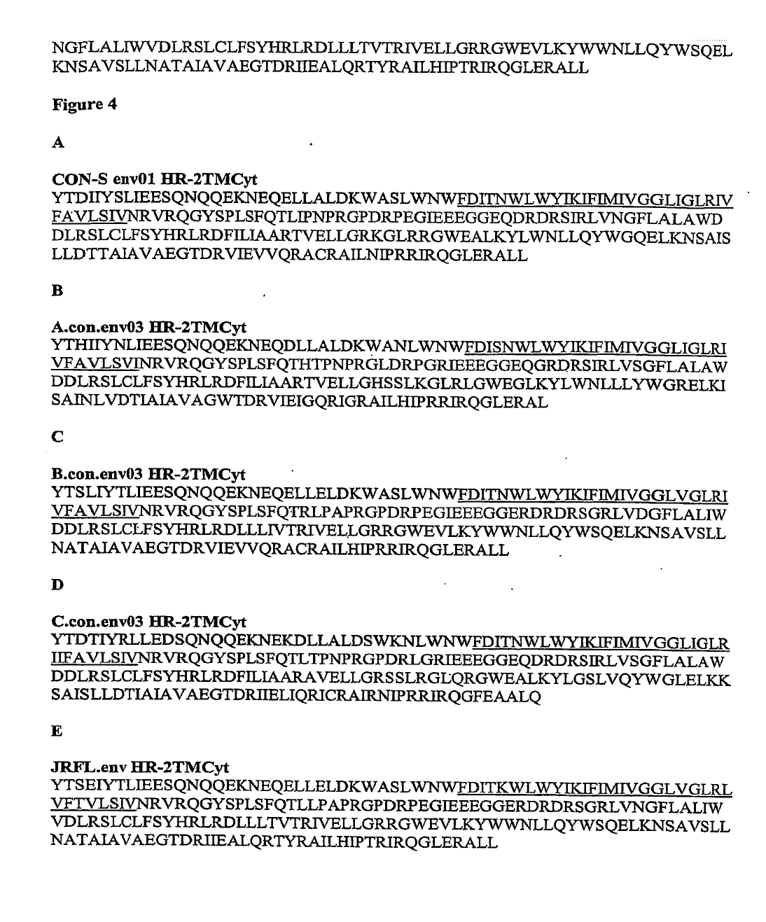

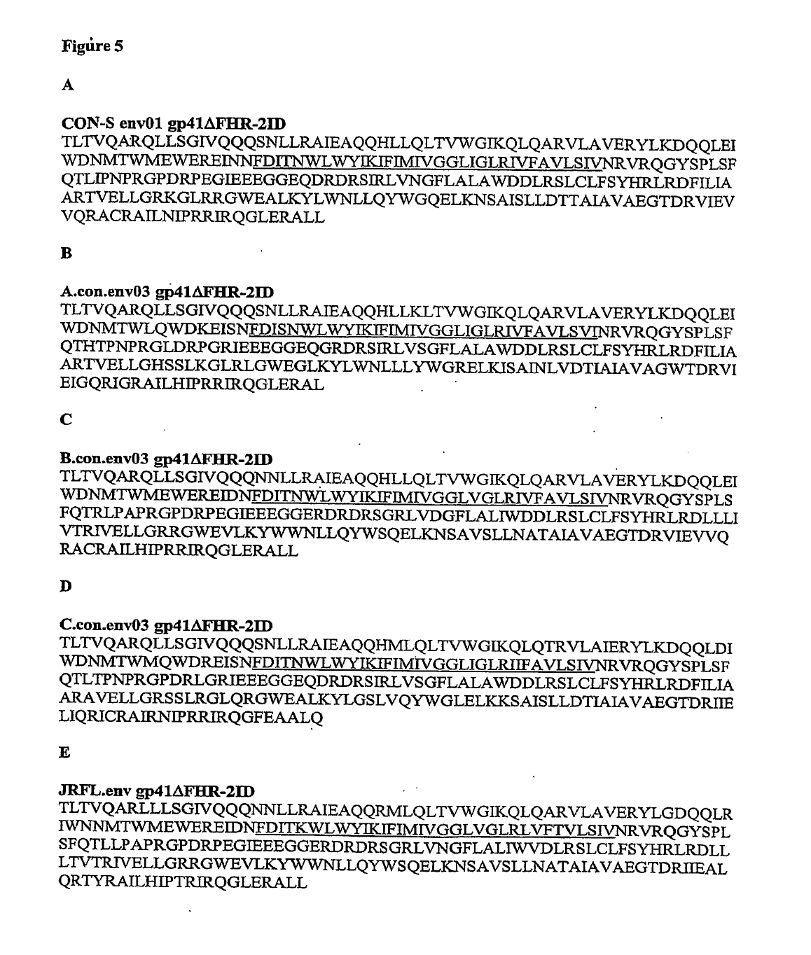

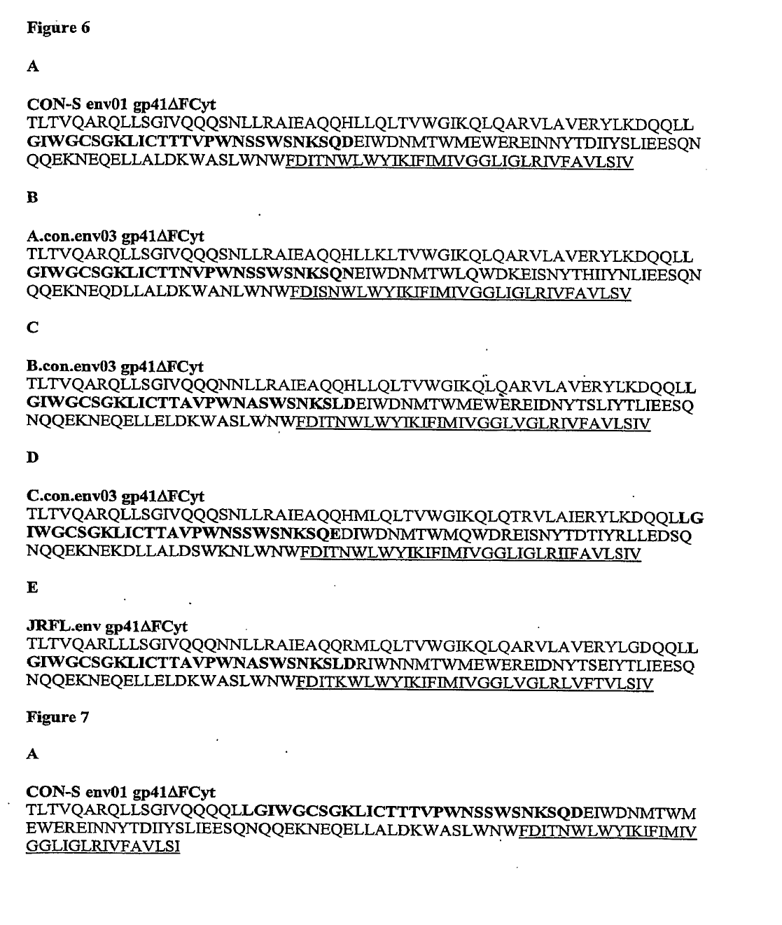

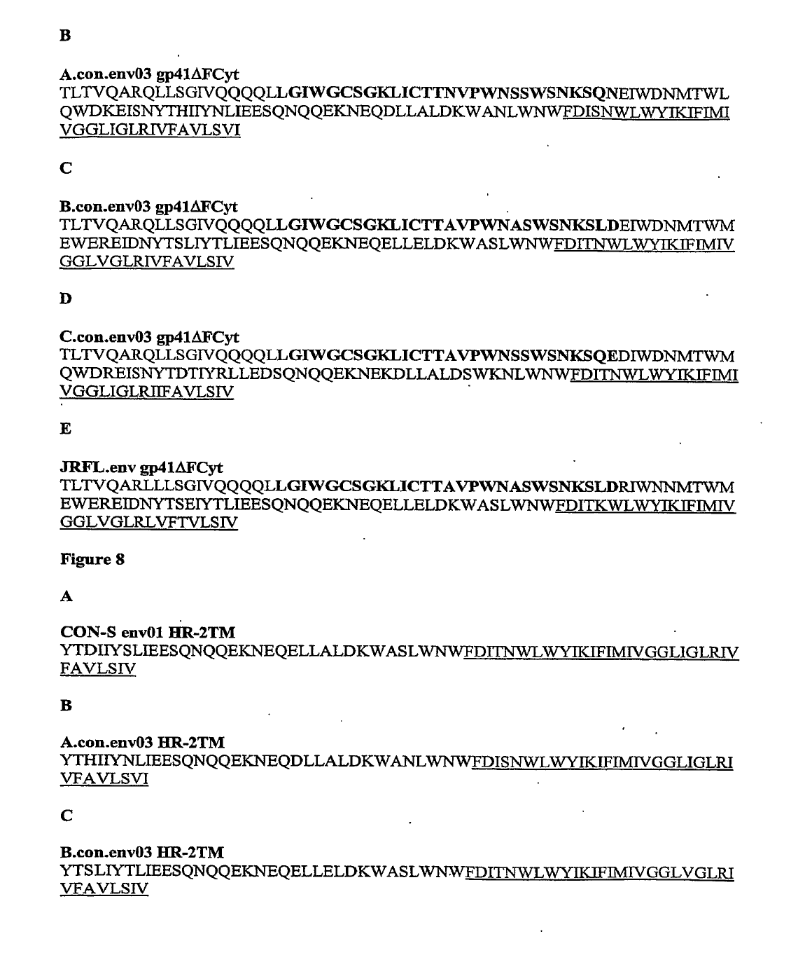

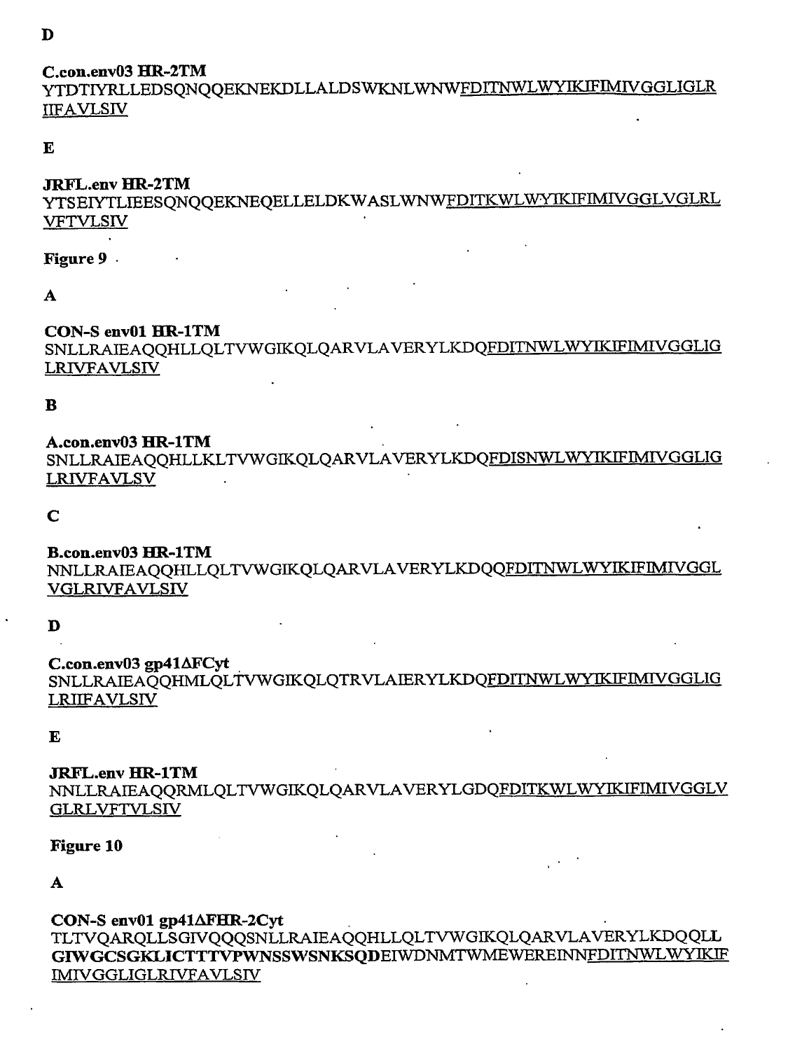

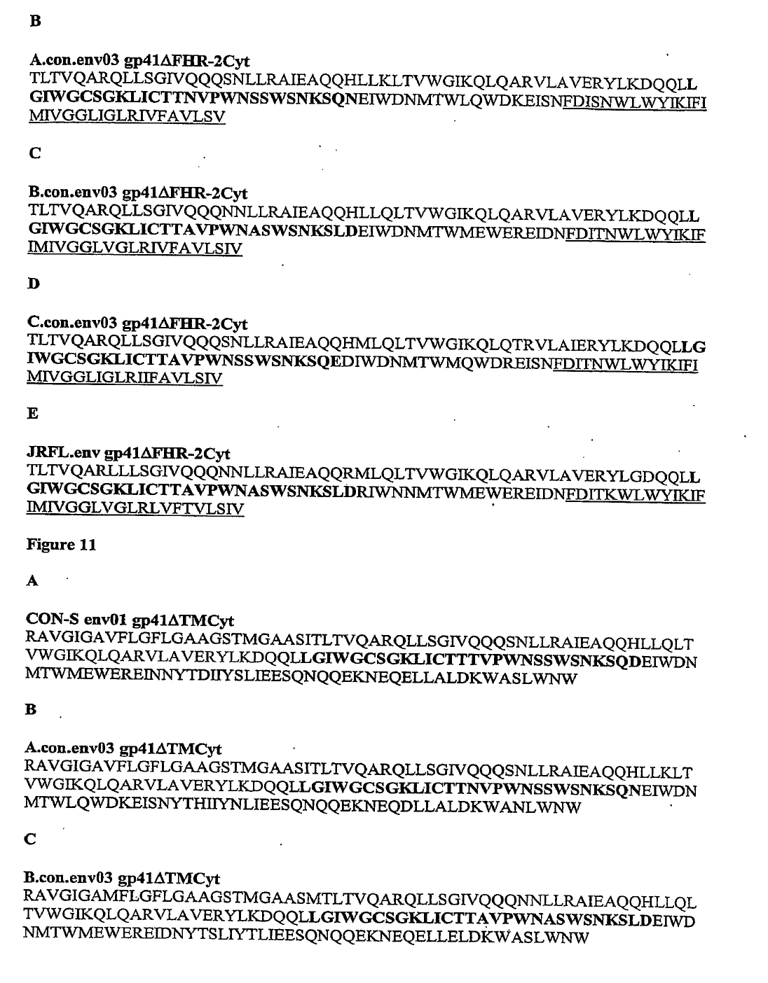

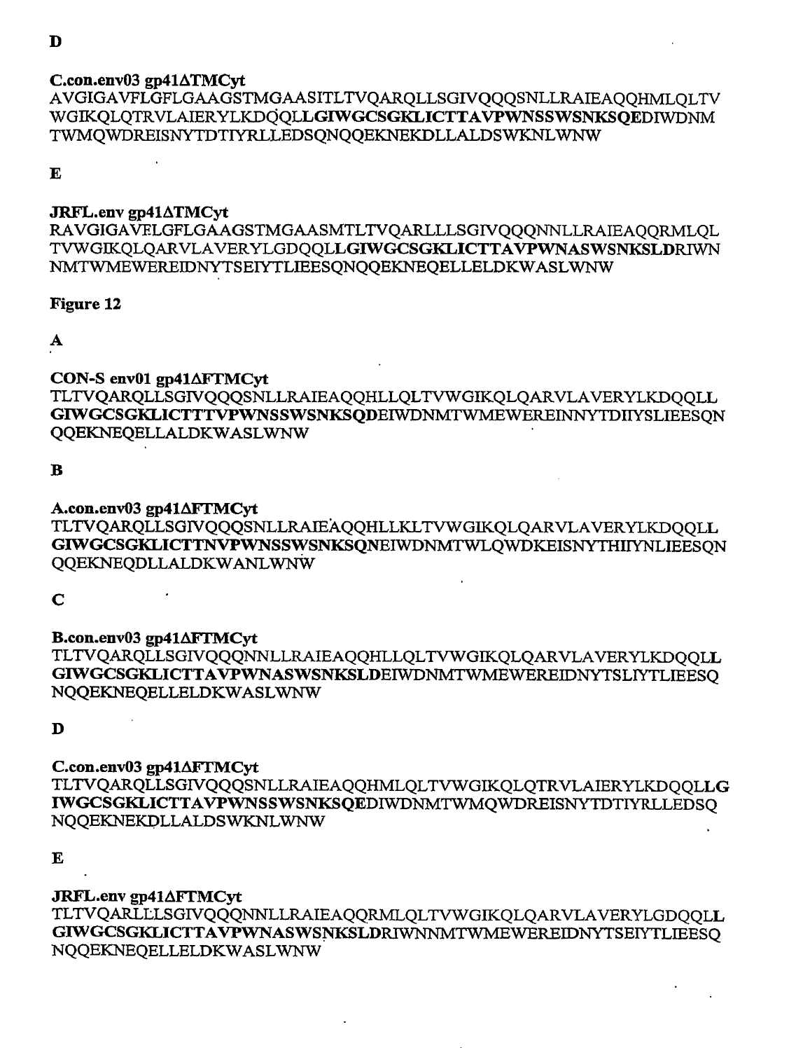

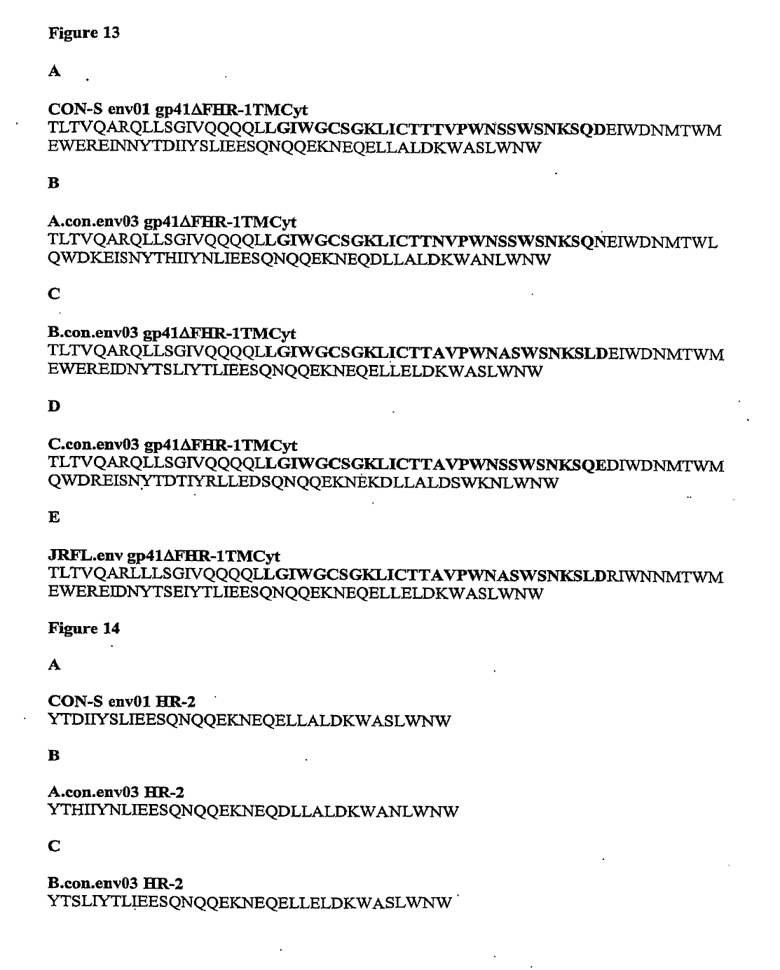

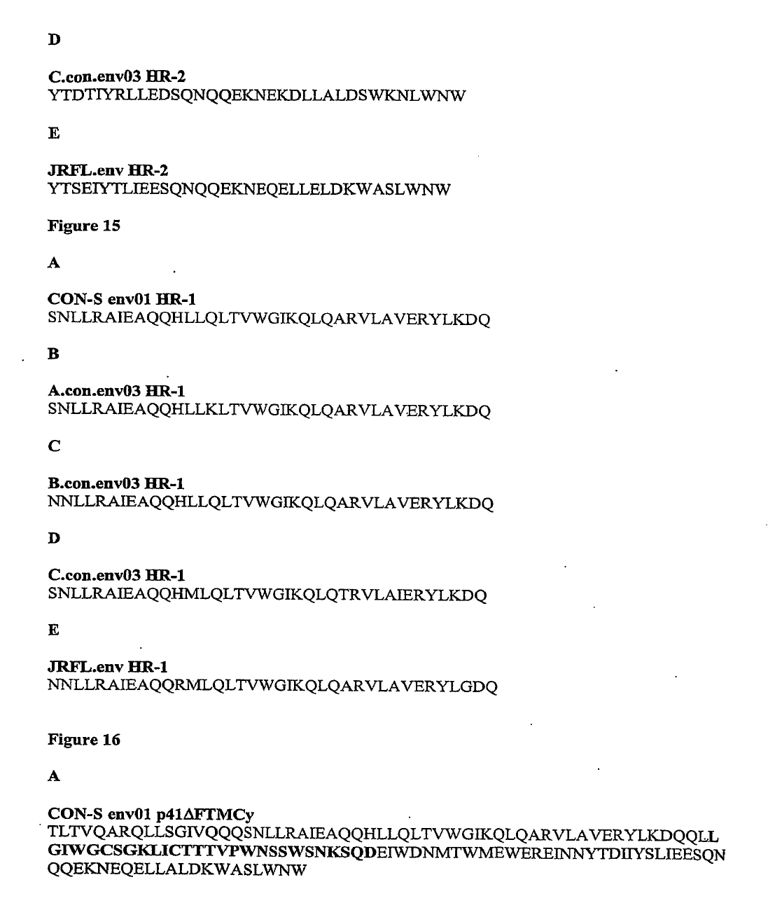

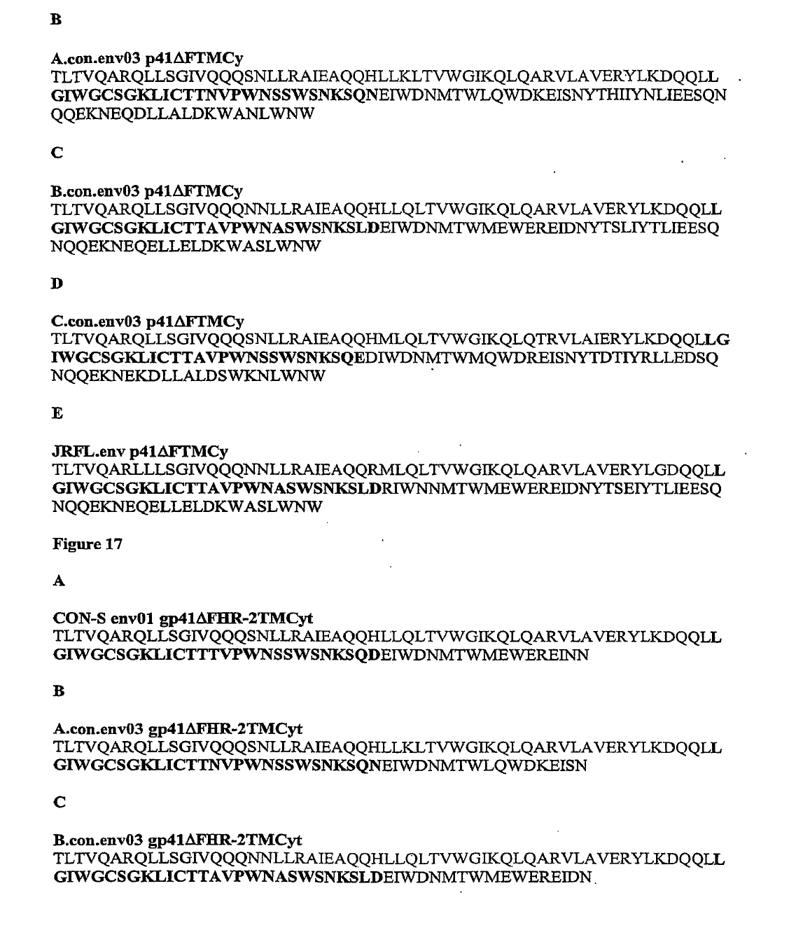

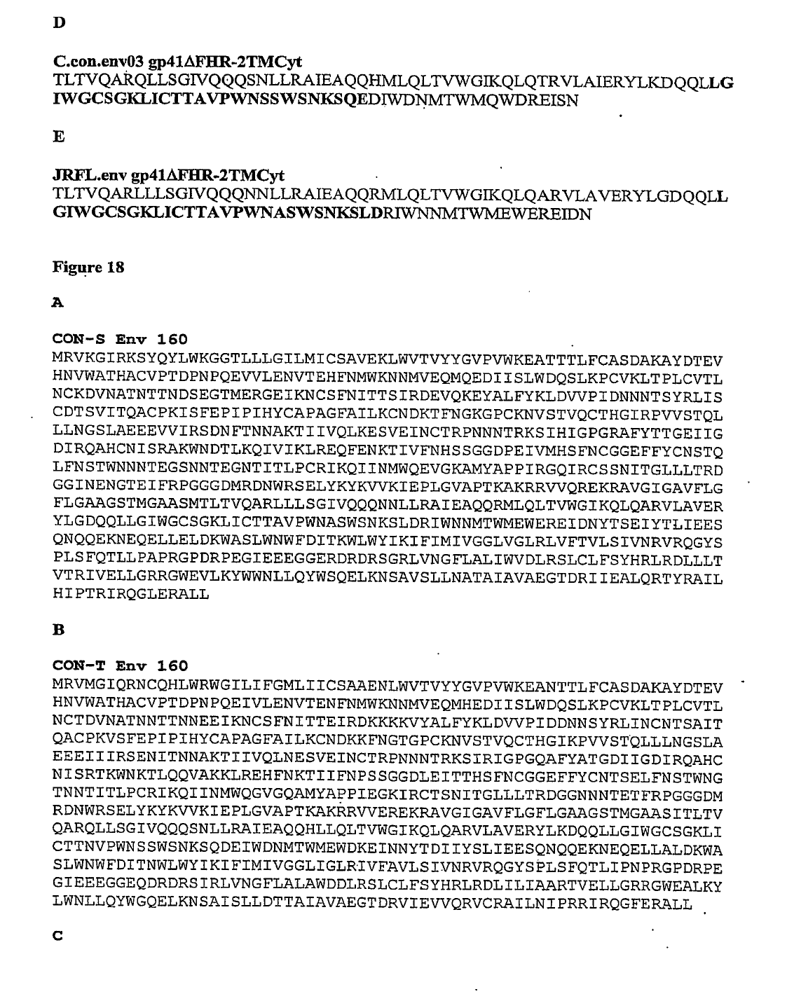

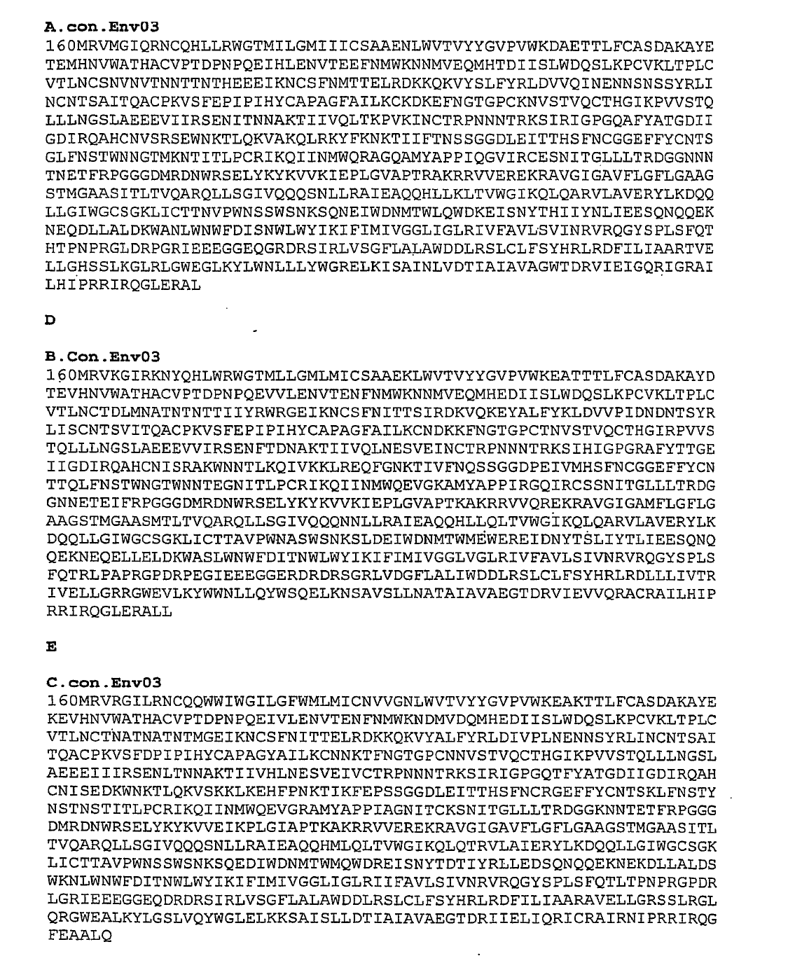

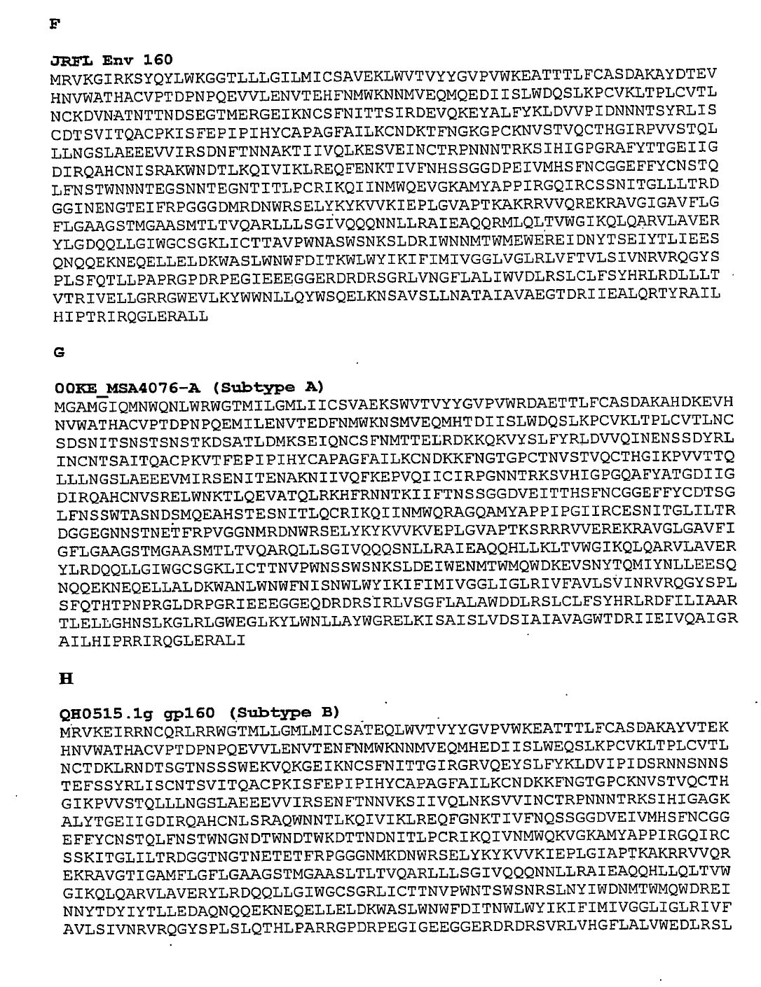

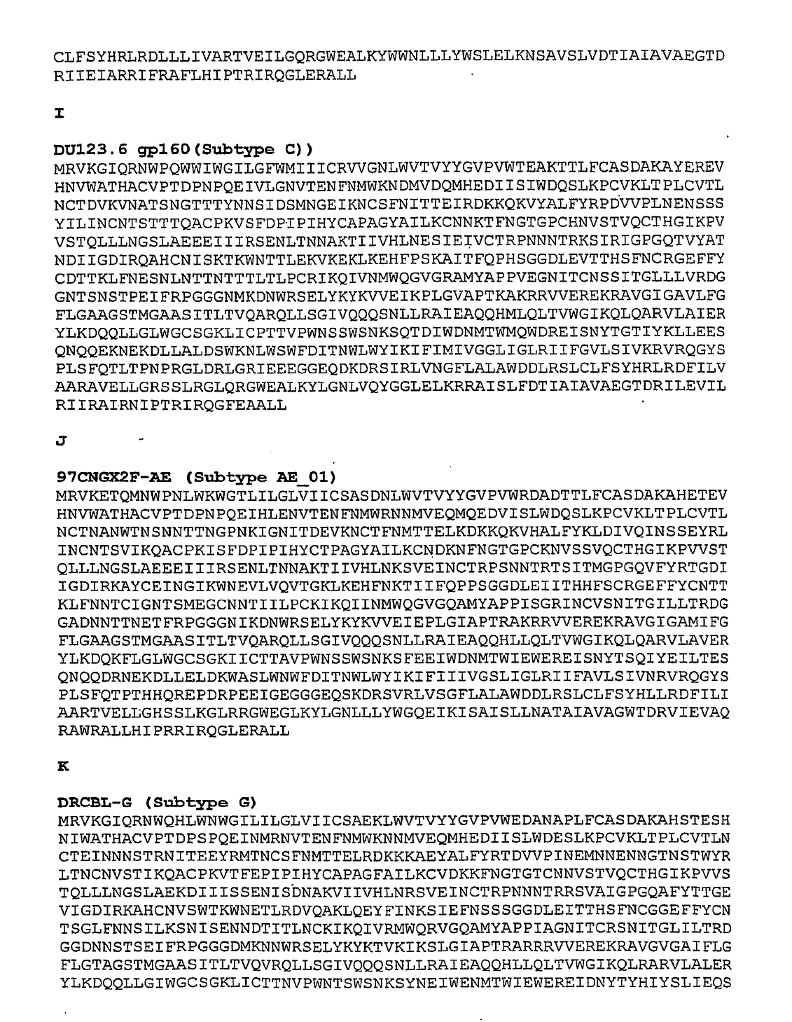

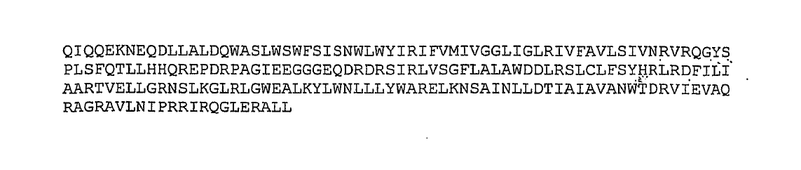

The present invention combines vaccination using, for example, one or more immunogens set forth in FIGS. 1-18 (or chimeras (e.g., HIV-2 envelop sequences containing HIV-1 MPER sequences) such as those described by Bibollet-Ruche and by Shaw at the AIDS Vaccine meeting in Montreal Sep. 8, 2005, (www.aidsvaccine05.org)), or nucleic acids encoding same, with a gene therapy approach that protects cells (e.g., rectal or vaginal mucosal cells) from viral challenge.

In the gene therapy aspect of the present invention, a nucleic acid sequence encoding a membrane-anchored peptide that inhibits HIV-1 entry into the cells at the level of membrane fusion can be introduced into the cells to be protected. The nucleic acid can be present in a vector, e.g., a viral vector. Administration of the vector is effected under conditions such that, upon introduction into the cells, the nucleic acid is expressed so that the cells display on their surface the fusion inhibitor. An administration regimen can be selected that ensures maintenance of the protective effect until such time as an effective immune response has been developed.

Examples of suitable membrane-anchored peptides include those set forth in the attached figures that comprise a transmembrane domain and a fusion inhibitory peptide. When nucleic acids encoding such peptides are administered, the nucleic acids are advantageously codon optimized. Appropriate vectors include those described below.

The vaccination aspect of the invention can be effected using one or more immunogens set forth in FIGS. 1-18, or nucleic acids (advantageously codon optimized) encoding same. Appropriate immunization strategies can be established by one skilled in the art (see, for example, strategies described in PCT/US04/30397).

The immunogen of the invention can be formulated with a pharmaceutically acceptable carrier and/or adjuvant (such as alum) using techniques well known in the art. Suitable routes of administration to effect immunization include systemic (e.g. intramuscular, subcutaneous, or intranasal). Suitable routes of administration to effect cell protection can vary with the cell type targeted for protection (for example, when mucosal cells are targeted for protection, administration can be, for example, vaginal or rectal).

The immunogens of the invention (peptide or nucleic acid) can be chemically synthesized and purified using methods which are well known to the ordinarily skilled artisan. The immunogens can also be synthesized by well-known recombinant DNA techniques. Nucleic acids encoding the immunogens of the invention can be used as components of, for example, a DNA vaccine wherein the encoding sequence is administered as naked DNA or, for example, a minigene encoding the immunogen can be present in a viral vector. The encoding sequence can be present, for example, in a replicating or non-replicating adenoviral vector, an adeno-associated virus vector, an attenuated mycobacterium tuberculosis vector, a Bacillus Calmette Guerin (BCG) vector, a vaccinia or Modified Vaccinia Ankara (MVA) vector, another pox virus vector, recombinant polio and other enteric virus vector, Salmonella species bacterial vector, Shigella species bacterial vector, Venezuelean Equine Encephalitis Virus (VEE) vector, a Semliki Forest Virus vector, a VSV vector or a Tobacco Mosaic Virus vector. The encoding sequence, can also be expressed as a DNA plasmid with, for example, an active promoter such as a CMV promoter. Other live vectors can also be used to express the sequences of the invention. Expression of the immunogen of the invention can be induced in a patient's own cells, by introduction into those cells of nucleic acids that encode the immunogen, preferably using codons and promoters that optimize expression in human cells. Examples of methods of making and using DNA vaccines are disclosed in U.S. Pat. Nos. 5,580,859, 5,589,466, and 5,703,055.

The composition of the invention comprises an immunologically effective amount of the immunogen of this invention, or nucleic acid sequence encoding same, in a pharmaceutically acceptable delivery system. The compositions can be used for prevention and/or treatment of immunodeficiency virus infection. The compositions of the invention can be formulated using adjuvants, emulsifiers, pharmaceutically-acceptable carriers or other ingredients routinely provided in vaccine compositions. Optimum formulations can be readily designed by one of ordinary skill in the art and can include formulations for immediate release and/or for sustained release, and for induction of systemic immunity and/or induction of localized mucosal immunity (e.g, the formulation can be designed for vaginal or rectal administration, e.g. as a suppository). The present compositions can be administered by any convenient route including subcutaneous, intranasal, oral, intramuscular, or other parenteral or enteral route, depending on the effect sought. The immunogens (or encoding nucleic acids) can be administered as a single dose or multiple doses. Optimum immunization schedules can be readily determined by the ordinarily skilled artisan and can vary with the patient, the composition and the effect sought. Adjuvants suitable for use in the present invention include those described in PCT/US05/37384.

The invention contemplates the direct use of both the immunogen of the invention and/or nucleic acids encoding same and/or the immunogen expressed as minigenes in the vectors indicated above. For example, a minigene encoding the immunogen can be used as a prime and/or boost.

The invention includes any and all amino acid sequences disclosed herein and, where applicable, CF and CFI forms thereof, as well as nucleic acid sequences encoding same (and nucleic acids complementary to such encoding sequences).

All documents and other information sources cited above are hereby incorporated herein by reference.

What is claimed is:

1. An isolated immunogen comprising an amino acid sequence as set forth in FIGS. 1-18.

2. A composition comprising at least 1 immunogen comprising an amino acid sequence as set forth in FIGS. 1-18 and a carrier.

3. An isolated nucleic acid encoding an immunogen comprising an amino acid sequence as set forth in FIGS. 1-18.

4. A construct comprising said nucleic acid according to claim 3 and a vector.

5. The construct according to claim 4 wherein said vector is a viral vector.

6. The construct according to claim 5 wherein said viral vector is an adenoviral vector, an adeno-associated viral vector, an attenuated mycobacterium tuberculosis vector, a Bacillus Calmette Guerin viral vector, or a vaccinia or Modified Vaccinia Ankara viral vector.

7. The construct according to claim 5 wherein said viral vector is pox virus vector or an enteric virus vector.

8. The construct according to claim 4 wherein said nucleic acid is operably linked to a promoter.

9. A minigene comprising a nucleic acid sequence encoding an immunogen as set forth in FIGS. 1-18.

10. A method of inducing an immune response in a patient and protecting said patient from viral challenge comprising administering to said patient an amount of an immunogen comprising an amino acid sequence as set forth in FIGS. 1-18 sufficient to induce said immune response and introducing into cells of said patient a nucleic acid sequence encoding a membrane-anchored peptide that inhibits human immunodeficiency virus-1 (HIV-1) entry into cells under conditions such that said nucleic acid sequence is expressed and said membrane-anchored peptide is displayed on the surface of said cells whereby entry of HIV-1 into said cells is inhibited and said patient is thereby protected from viral challenge.

11. The method according to claim 10 wherein said membrane-anchored peptide comprises a transmembrane domain and a fusion inhibitory protein.


SEQUENCE LISTING (98 sequences)

SEQ ID NO: 1 - DNA, 102 bp, Artificial Sequence
  Description of Artificial Sequence: Synthetic polynucleotide
tctagagccgccatgcgcgtgcgcggcatccagcgcaactgccagcacctgtggcgctggggcaccctgatcctgggcatgctgatgatctgctccgccgcc

SEQ ID NO: 2 - protein, 30 aa, Artificial Sequence
  Description of Artificial Sequence: Synthetic polypeptide
MRVRGIQRNCQHLWRWGTLILGMLMICSAA

SEQ ID NO: 3 - protein, 350 aa, Artificial Sequence
  Description of Artificial Sequence: Synthetic polypeptide
RAVGIGAVFLGFLGAAGSTMGAASITLTVQARQLLSGIVQQQSNLLRAIEAQQHLLQLTVWGIKQLQARVLAVERYLKDQQLLGIWGCSGKLICTTTVPWNSSWSNKSQDEIWDNMTWMEWEREINNYTDIIYSLIEESQNQQEKNEQELLALDKWASLWNWFDITNWLWYIKIFIMIVGGLIGLRIVFAVLSIVNRVRQGYSPLSFQTLIPNPRGPDRPEGIEEEGGEQDRDRSIRLVNGFLALAWDDLRSLCLFSYHRLRDFILIAARTVELLGRKGLRRGWEALKYLWNLLQYWGQELKNSAISLLDTTAIAVAEGTDRVIEVVQRACRAILNIPRRIRQGLERALL

SEQ ID NO: 4 - protein, 352 aa, Artificial Sequence
  Description of Artificial Sequence: Synthetic polypeptide
RAVGIGAVFLGFLGAAGSTMGAASITLTVQARQLLSGIVQQQSNLLRAIEAQQHLLKLTVWGIKQLQARVLAVERYLKDQQLLGIWGCSGKLICTTNVPWNSSWSNKSQNEIWDNMTWLQWDKEISNYTHIIYNLIEESQNQQEKNEQDLLALDKWANLWNWFDISNWLWYIKIFIMIVGGLIGLRIVFAVLSVINRVRQGYSPLSFQTHTPNPRGLDRPGRIEEEGGEQGRDRSIRLVSGFLALAWDDLRSLCLFSYHRLRDFILIAARTVELLGHSSLKGLRLGWEGLKYLWNLLLYWGRELKISAINLVDTIAIAVAGWTDRVIEIGQRIGRAILHIPRRIRQGLERAL

SEQ ID NO: 5 - protein, 346 aa, Artificial Sequence
  Description of Artificial Sequence: Synthetic polypeptide
RAVGIGAMFLGFLGAAGSTMGAASMTLTVQARQLLSGIVQQQNNLLRAIEAQQHLLQLTVWGIKQLQARVLAVERYLKDQQLLGIWGCSGKLICTTAVPWNASWSNKSLDEIWDNMTWMEWEREIDNYTSLIYTLIEESQNQQEKNEQELLELDKWASLWNWFDITNWLWYIKIFIMIVGGLVGLRIVFAVLSIVNRVRQGYSPLSFQTRLPAPRGPDRPEGIEEEGGERDRDRSGRLVDGFLALIWDDLRSLCLFSYHRLRDLLLIVTRIVELLGRRGWEVLKYWWNLLQYWSQELKNSAVSLLNATAIAVAEGTDRVIEVVQRACRAILHIPRRIRQGLERALL

SEQ ID NO: 6 - protein, 352 aa, Artificial Sequence
  Description of Artificial Sequence: Synthetic polypeptide
AVGIGAVFLGFLGAAGSTMGAASITLTVQARQLLSGIVQQQSNLLRAIEAQQHMLQLTVWGIKQLQTRVLAIERYLKDQQLLGIWGCSGKLICTTAVPWNSSWSNKSQEDIWDNMTWMQWDREISNYTDTIYRLLEDSQNQQEKNEKDLLALDSWKNLWNWFDITNWLWYIKIFIMIVGGLIGLRIIFAVLSIVNRVRQGYSPLSFQTLTPNPRGPDRLGRIEEEGGEQDRDRSIRLVSGFLALAWDDLRSLCLFSYHRLRDFILIAARAVELLGRSSLRGLQRGWEALKYLGSLVQYWGLELKKSAISLLDTIAIAVAEGTDRIIELIQRICRAIRNIPRRIRQGFEAALQ

SEQ ID NO: 7 - protein, 346 aa, Artificial Sequence
  Description of Artificial Sequence: Synthetic polypeptide
RAVGIGAVFLGFLGAAGSTMGAASMTLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDNYTSEIYTLIEESQNQQEKNEQELLELDKWASLWNWFDITKWLWYIKIFIMIVGGLVGLRLVFTVLSIVNRVRQGYSPLSFQTLLPAPRGPDRPEGIEEEGGERDRDRSGRLVNGFLALIWVDLRSLCLFSYHRLRDLLLTVTRIVELLGRRGWEVLKYWWNLLQYWSQELKNSAVSLLNATAIAVAEGTDRIIEALQRTYRAILHIPTRIRQGLERALL

SEQ ID NO: 8 - protein, 325 aa, Artificial Sequence
  Description of Artificial Sequence: Synthetic polypeptide
TLTVQARQLLSGIVQQQSNLLRAIEAQQHLLQLTVWGIKQLQARVLAVERYLKDQQLLGIWGCSGKLICTTTVPWNSSWSNKSQDEIWDNMTWMEWEREINNYTDIIYSLIEESQNQQEKNEQELLALDKWASLWNWFDITNWLWYIKIFIMIVGGLIGLRIVFAVLSIVNRVRQGYSPLSFQTLIPNPRGPDRPEGIEEEGGEQDRDRSIRLVNGFLALAWDDLRSLCLFSYHRLRDFILIAARTVELLGRKGLRRGWEALKYLWNLLQYWGQELKNSAISLLDTTAIAVAEGTDRVIEVVQRACRAILNIPRRIRQGLERALL

SEQ ID NO: 9 - protein, 327 aa, Artificial Sequence
  Description of Artificial Sequence: Synthetic polypeptide
TLTVQARQLLSGIVQQQSNLLRAIEAQQHLLKLTVWGIKQLQARVLAVERYLKDQQLLGIWGCSGKLICTTNVPWNSSWSNKSQNEIWDNMTWLQWDKEISNYTHIIYNLIEESQNQQEKNEQDLLALDKWANLWNWFDISNWLWYIKIFIMIVGGLIGLRIVFAVLSVINRVRQGYSPLSFQTHTPNPRGLDRPGRIEEEGGEQGRDRSIRLVSGFLALAWDDLRSLCLFSYHRLRDFILIAARTVELLGHSSLKGLRLGWEGLKYLWNLLLYWGRELKISAINLVDTIAIAVAGWTDRVIEIGQRIGRAILHIPRRIRQGLERAL

SEQ ID NO: 10 - protein, 321 aa, Artificial Sequence
  Description of Artificial Sequence: Synthetic polypeptide
TLTVQARQLLSGIVQQQNNLLRAIEAQQHLLQLTVWGIKQLQARVLAVERYLKDQQLLGIWGCSGKLICTTAVPWNASWSNKSLDEIWDNMTWMEWEREIDNYTSLIYTLIEESQNQQEKNEQELLELDKWASLWNWFDITNWLWYIKIFIMIVGGLVGLRIVFAVLSIVNRVRQGYSPLSFQTRLPAPRGPDRPEGIEEEGGERDRDRSGRLVDGFLALIWDDLRSLCLFSYHRLRDLLLIVTRIVELLGRRGWEVLKYWWNLLQYWSQELKNSAVSLLNATAIAVAEGTDRVIEVVQRACRAILHIPRRIRQGLERALL

SEQ ID NO: 11 - protein, 328 aa, Artificial Sequence
  Description of Artificial Sequence: Synthetic polypeptide
TLTVQARQLLSGIVQQQSNLLRAIEAQQHMLQLTVWGIKQLQTRVLAIERYLKDQQLLGIWGCSGKLICTTAVPWNSSWSNKSQEDIWDNMTWMQWDREISNYTDTIYRLLEDSQNQQEKNEKDLLALDSWKNLWNWFDITNWLWYIKIFIMIVGGLIGLRIIFAVLSIVNRVRQGYSPLSFQTLTPNPRGPDRLGRIEEEGGEQDRDRSIRLVSGFLALAWDDLRSLCLFSYHRLRDFILIAARAVELLGRSSLRGLQRGWEALKYLGSLVQYWGLELKKSAISLLDTIAIAVAEGTDRIIELIQRICRAIRNIPRRIRQGFEAALQ

SEQ ID NO: 12 - protein, 321 aa, Artificial Sequence
  Description of Artificial Sequence: Synthetic polypeptide
TLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDNYTSEIYTLIEESQNQQEKNEQELLELDKWASLWNWFDITKWLWYIKIFIMIVGGLVGLRLVFTVLSIVNRVRQGYSPLSFQTLLPAPRGPDRPEGIEEEGGERDRDRSGRLVNGFLALIWVDLRSLCLFSYHRLRDLLLTVTRIVELLGRRGWEVLKYWWNLLQYWSQELKNSAVSLLNATAIAVAEGTDRIIEALQRTYRAILHIPTRIRQGLERALL

SEQ ID NO: 13 - protein, 287 aa, Artificial Sequence
  Description of Artificial Sequence: Synthetic polypeptide
TLTVQARQLLSGIVQQQQLLGIWGCSGKLICTTTVPWNSSWSNKSQDEIWDNMTWMEWEREINNYTDIIYSLIEESQNQQEKNEQELLALDKWASLWNWFDITNWLWYIKIFIMIVGGLIGLRIVFAVLSIVNRVRQGYSPLSFQTLIPNPRGPDRPEGIEEEGGEQDRDRSIRLVNGFLALAWDDLRSLCLFSYHRLRDFILIAARTVELLGRKGLRRGWEALKYLWNLLQYWGQELKNSAISLLDTTAIAVAEGTDRVIEVVQRACRAILNIPRRIRQGLERALL

SEQ ID NO: 14 - protein, 289 aa, Artificial Sequence
  Description of Artificial Sequence: Synthetic polypeptide
TLTVQARQLLSGIVQQQQLLGIWGCSGKLICTTNVPWNSSWSNKSQNEIWDNMTWLQWDKEISNYTHIIYNLIEESQNQQEKNEQDLLALDKWANLWNWFDISNWLWYIKIFIMIVGGLIGLRIVFAVLSVINRVRQGYSPLSFQTHTPNPRGLDRPGRIEEEGGEQGRDRSIRLVSGFLALAWDDLRSLCLFSYHRLRDFILIAARTVELLGHSSLKGLRLGWEGLKYLWNLLLYWGRELKISAINLVDTIAIAVAGWTDRVIEIGQRIGRAILHIPRRIRQGLERAL

SEQ ID NO: 15 - protein, 283 aa, Artificial Sequence
  Description of Artificial Sequence: Synthetic polypeptide
TLTVQARQLLSGIVQQQQLLGIWGCSGKLICTTAVPWNASWSNKSLDEIWDNMTWMEWEREIDNYTSLIYTLIEESQNQQEKNEQELLELDKWASLWNWFDITNWLWYIKIFIMIVGGLVGLRIVFAVLSIVNRVRQGYSPLSFQTRLPAPRGPDRPEGIEEEGGERDRDRSGRLVDGFLALIWDDLRSLCLFSYHRLRDLLLIVTRIVELLGRRGWEVLKYWWNLLQYWSQELKNSAVSLLNATAIAVAEGTDRVIEVVQRACRAILHIPRRIRQGLERALL

SEQ ID NO: 16 - protein, 290 aa, Artificial Sequence
  Description of Artificial Sequence: Synthetic polypeptide
TLTVQARQLLSGIVQQQQLLGIWGCSGKLICTTAVPWNSSWSNKSQEDIWDNMTWMQWDREISNYTDTIYRLLEDSQNQQEKNEKDLLALDSWKNLWNWFDITNWLWYIKIFIMIVGGLIGLRIIFAVLSIVNRVRQGYSPLSFQTLTPNPRGPDRLGRIEEEGGEQDRDRSIRLVSGFLALAWDDLRSLCLFSYHRLRDFILIAARAVELLGRSSLRGLQRGWEALKYLGSLVQYWGLELKKSAISLLDTIAIAVAEGTDRIIELIQRICRAIRNIPRRIRQGFEAALQ

SEQ ID NO: 17 - protein, 283 aa, Artificial Sequence
  Description of Artificial Sequence: Synthetic polypeptide
TLTVQARLLLSGIVQQQQLLGIWGCSGKLICTTAVPWNASWSNKSLDRIWNNMTWMEWEREIDNYTSEIYTLIEESQNQQEKNEQELLELDKWASLWNWFDITKWLWYIKIFIMIVGGLVGLRLVFTVLSIVNRVRQGYSPLSFQTLLPAPRGPDRPEGIEEEGGERDRDRSGRLVNGFLALIWVDLRSLCLFSYHRLRDLLLTVTRIVELLGRRGWEVLKYWWNLLQYWSQELKNSAVSLLNATAIAVAEGTDRIIEALQRTYRAILHIPTRIRQGLERALL

SEQ ID NO: 18 - protein, 223 aa, Artificial Sequence
  Description of Artificial Sequence: Synthetic polypeptide
YTDIIYSLIEESQNQQEKNEQELLALDKWASLWNWFDITNWLWYIKIFIMIVGGLIGLRIVFAVLSIVNRVRQGYSPLSFQTLIPNPRGPDRPEGIEEEGGEQDRDRSIRLVNGFLALAWDDLRSLCLFSYHRLRDFILIAARTVELLGRKGLRRGWEALKYLWNLLQYWGQELKNSAISLLDTTAIAVAEGTDRVIEVVQRACRAILNIPRRIRQGLERALL

SEQ ID NO: 19 - protein, 225 aa, Artificial Sequence
  Description of Artificial Sequence: Synthetic polypeptide
YTHIIYNLIEESQNQQEKNEQDLLALDKWANLWNWFDISNWLWYIKIFIMIVGGLIGLRIVFAVLSVINRVRQGYSPLSFQTHTPNPRGLDRPGRIEEEGGEQGRDRSIRLVSGFLALAWDDLRSLCLFSYHRLRDFILIAARTVELLGHSSLKGLRLGWEGLKYLWNLLLYWGRELKISAINLVDTIAIAVAGWTDRVIEIGQRIGRAILHIPRRIRQGLERAL

SEQ ID NO: 20 - protein, 219 aa, Artificial Sequence
  Description of Artificial Sequence: Synthetic polypeptide
YTSLIYTLIEESQNQQEKNEQELLELDKWASLWNWFDITNWLWYIKIFIMIVGGLVGLRIVFAVLSIVNRVRQGYSPLSFQTRLPAPRGPDRPEGIEEEGGERDRDRSGRLVDGFLALIWDDLRSLCLFSYHRLRDLLLIVTRIVELLGRRGWEVLKYWWNLLQYWSQELKNSAVSLLNATAIAVAEGTDRVIEVVQRACRAILHIPRRIRQGLERALL

SEQ ID NO: 21 - protein, 226 aa, Artificial Sequence
  Description of Artificial Sequence: Synthetic polypeptide
YTDTIYRLLEDSQNQQEKNEKDLLALDSWKNLWNWFDITNWLWYIKIFIMIVGGLIGLRIIFAVLSIVNRVRQGYSPLSFQTLTPNPRGPDRLGRIEEEGGEQDRDRSIRLVSGFLALAWDDLRSLCLFSYHRLRDFILIAARAVELLGRSSLRGLQRGWEALKYLGSLVQYWGLELKKSAISLLDTIAIAVAEGTDRIIELIQRICRAIRNIPRRIRQGFEAALQ

SEQ ID NO: 22 - protein, 219 aa, Artificial Sequence
  Description of Artificial Sequence: Synthetic polypeptide
YTSEIYTLIEESQNQQEKNEQELLELDKWASLWNWFDITKWLWYIKIFIMIVGGLVGLRLVFTVLSIVNRVRQGYSPLSFQTLLPAPRGPDRPEGIEEEGGERDRDRSGRLVNGFLALIWVDLRSLCLFSYHRLRDLLLTVTRIVELLGRRGWEVLKYWWNLLQYWSQELKNSAVSLLNATAIAVAEGTDRIIEALQRTYRAILHIPTRIRQGLERALL

SEQ ID NO: 23 - protein, 262 aa, Artificial Sequence
  Description of Artificial Sequence: Synthetic polypeptide
TLTVQARQLLSGIVQQQSNLLRAIEAQQHLLQLTVWGIKQLQARVLAVERYLKDQQLEIWDNMTWMEWEREINNFDITNWLWYIKIFIMIVGGLIGLRIVFAVLSIVNRVRQGYSPLSFQTLIPNPRGPDRPEGIEEEGGEQDRDRSIRLVNGFLALAWDDLRSLCLFSYHRLRDFILIAARTVELLGRKGLRRGWEALKYLWNLLQYWGQELKNSAISLLDTTAIAVAEGTDRVIEVVQRACRAILNIPRRIRQGLERALL

SEQ ID NO: 24 - protein, 264 aa, Artificial Sequence
  Description of Artificial Sequence: Synthetic polypeptide
TLTVQARQLLSGIVQQQSNLLRAIEAQQHLLKLTVWGIKQLQARVLAVERYLKDQQLEIWDNMTWLQWDKEISNFDISNWLWYIKIFIMIVGGLIGLRIVFAVLSVINRVRQGYSPLSFQTHTPNPRGLDRPGRIEEEGGEQGRDRSIRLVSGFLALAWDDLRSLCLFSYHRLRDFILIAARTVELLGHSSLKGLRLGWEGLKYLWNLLLYWGRELKISAINLVDTIAIAVAGWTDRVIEIGQRIGRAILHIPRRIRQGLERAL

SEQ ID NO: 25 - protein, 258 aa, Artificial Sequence
  Description of Artificial Sequence: Synthetic polypeptide
TLTVQARQLLSGIVQQQNNLLRAIEAQQHLLQLTVWGIKQLQARVLAVERYLKDQQLEIWDNMTWMEWEREIDNFDITNWLWYIKIFIMIVGGLVGLRIVFAVLSIVNRVRQGYSPLSFQTRLPAPRGPDRPEGIEEEGGERDRDRSGRLVDGFLALIWDDLRSLCLFSYHRLRDLLLIVTRIVELLGRRGWEVLKYWWNLLQYWSQELKNSAVSLLNATAIAVAEGTDRVIEVVQRACRAILHIPRRIRQGLERALL

SEQ ID NO: 26 - protein, 265 aa, Artificial Sequence
  Description of Artificial Sequence: Synthetic polypeptide
TLTVQARQLLSGIVQQQSNLLRAIEAQQHMLQLTVWGIKQLQTRVLAIERYLKDQQLDIWDNMTWMQWDREISNFDITNWLWYIKIFIMIVGGLIGLRIIFAVLSIVNRVRQGYSPLSFQTLTPNPRGPDRLGRIEEEGGEQDRDRSIRLVSGFLALAWDDLRSLCLFSYHRLRDFILIAARAVELLGRSSLRGLQRGWEALKYLGSLVQYWGLELKKSAISLLDTIAIAVAEGTDRIIELIQRICRAIRNIPRRIRQGFEAALQ

SEQ ID NO: 27 - protein, 258 aa, Artificial Sequence
  Description of Artificial Sequence: Synthetic polypeptide
TLTVQARLLLSGIVQQQNNLLRAIEAQQRMLQLTVWGIKQLQARVLAVERYLGDQQLRIWNNMTWMEWEREIDNFDITKWLWYIKIFIMIVGGLVGLRLVFTVLSIVNRVRQGYSPLSFQTLLPAPRGPDRPEGIEEEGGERDRDRSGRLVNGFLALIWVDLRSLCLFSYHRLRDLLLTVTRIVELLGRRGWEVLKYWWNLLQYWSQELKNSAVSLLNATAIAVAEGTDRIIEALQRTYRAILHIPTRIRQGLERALL

SEQ ID NO: 28 - protein, 170 aa, Artificial Sequence
  Description of Artificial Sequence: Synthetic polypeptide
TLTVQARQLLSGIVQQQSNLLRAIEAQQHLLQLTVWGIKQLQARVLAVERYLKDQQLLGIWGCSGKLICTTTVPWNSSWSNKSQDEIWDNMTWMEWEREINNYTDIIYSLIEESQNQQEKNEQELLALDKWASLWNWFDITNWLWYIKIFIMIVGGLIGLRIVFAVLSIV

SEQ ID NO: 29 - protein, 169 aa, Artificial Sequence
  Description of Artificial Sequence: Synthetic polypeptide
TLTVQARQLLSGIVQQQSNLLRAIEAQQHLLKLTVWGIKQLQARVLAVERYLKDQQLLGIWGCSGKLICTTNVPWNSSWSNKSQNEIWDNMTWLQWDKEISNYTHIIYNLIEESQNQQEKNEQDLLALDKWANLWNWFDISNWLWYIKIFIMIVGGLIGLRIVFAVLSV

SEQ ID NO: 30 - protein, 170 aa, Artificial Sequence
  Description of Artificial Sequence: Synthetic polypeptide
TLTVQARQLLSGIVQQQNNLLRAIEAQQHLLQLTVWGIKQLQARVLAVERYLKDQQLLGIWGCSGKLICTTAVPWNASWSNKSLDEIWDNMTWMEWEREIDNYTSLIYTLIEESQNQQEKNEQELLELDKWASLWNWFDITNWLWYIKIFIMIVGGLVGLRIVFAVLSIV

SEQ ID NO: 31 - protein, 170 aa, Artificial Sequence
  Description of Artificial Sequence: Synthetic polypeptide
TLTVQARQLLSGIVQQQSNLLRAIEAQQHMLQLTVWGIKQLQTRVLAIERYLKDQQLLGIWGCSGKLICTTAVPWNSSWSNKSQEDIWDNMTWMQWDREISNYTDTIYRLLEDSQNQQEKNEKDLLALDSWKNLWNWFDITNWLWYIKIFIMIVGGLIGLRIIFAVLSIV

SEQ ID NO: 32 - protein, 170 aa, Artificial Sequence
  Description of Artificial Sequence: Synthetic polypeptide
TLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDNYTSEIYTLIEESQNQQEKNEQELLELDKWASLWNWFDITKWLWYIKIFIMIVGGLVGLRLVFTVLSIV

SEQ ID NO: 33 - protein, 131 aa, Artificial Sequence
  Description of Artificial Sequence: Synthetic polypeptide
TLTVQARQLLSGIVQQQQLLGIWGCSGKLICTTTVPWNSSWSNKSQDEIWDNMTWMEWEREINNYTDIIYSLIEESQNQQEKNEQELLALDKWASLWNWFDITNWLWYIKIFIMIVGGLIGLRIVFAVLSI

SEQ ID NO: 34 - protein, 132 aa, Artificial Sequence
  Description of Artificial Sequence: Synthetic polypeptide
TLTVQARQLLSGIVQQQQLLGIWGCSGKLICTTNVPWNSSWSNKSQNEIWDNMTWLQWDKEISNYTHIIYNLIEESQNQQEKNEQDLLALDKWANLWNWFDISNWLWYIKIFIMIVGGLIGLRIVFAVLSVI

SEQ ID NO: 35 - protein, 132 aa, Artificial Sequence
  Description of Artificial Sequence: Synthetic polypeptide
TLTVQARQLLSGIVQQQQLLGIWGCSGKLICTTAVPWNASWSNKSLDEIWDNMTWMEWEREIDNYTSLIYTLIEESQNQQEKNEQELLELDKWASLWNWFDITNWLWYIKIFIMIVGGLVGLRIVFAVLSIV

SEQ ID NO: 36 - protein, 132 aa, Artificial Sequence
  Description of Artificial Sequence: Synthetic polypeptide
TLTVQARQLLSGIVQQQQLLGIWGCSGKLICTTAVPWNSSWSNKSQEDIWDNMTWMQWDREISNYTDTIYRLLEDSQNQQEKNEKDLLALDSWKNLWNWFDITNWLWYIKIFIMIVGGLIGLRIIFAVLSIV

SEQ ID NO: 37 - protein, 132 aa, Artificial Sequence
  Description of Artificial Sequence: Synthetic polypeptide
TLTVQARLLLSGIVQQQQLLGIWGCSGKLICTTAVPWNASWSNKSLDRIWNNMTWMEWEREIDNYTSEIYTLIEESQNQQEKNEQELLELDKWASLWNWFDITKWLWYIKIFIMIVGGLVGLRLVFTVLSIV

SEQ ID NO: 38 - protein, 68 aa, Artificial Sequence
  Description of Artificial Sequence: Synthetic polypeptide
YTDIIYSLIEESQNQQEKNEQELLALDKWASLWNWFDITNWLWYIKIFIMIVGGLIGLRIVFAVLSIV

SEQ ID NO: 39 - protein, 68 aa, Artificial Sequence
  Description of Artificial Sequence: Synthetic polypeptide
YTHIIYNLIEESQNQQEKNEQDLLALDKWANLWNWFDISNWLWYIKIFIMIVGGLIGLRIVFAVLSVI

SEQ ID NO: 40 - protein, 68 aa, Artificial Sequence
  Description of Artificial Sequence: Synthetic polypeptide
YTSLIYTLIEESQNQQEKNEQELLELDKWASLWNWFDITNWLWYIKIFIMIVGGLVGLRIVFAVLSIV

SEQ ID NO: 41 - protein, 68 aa, Artificial Sequence
  Description of Artificial Sequence: Synthetic polypeptide
YTDTIYRLLEDSQNQQEKNEKDLLALDSWKNLWNWFDITNWLWYIKIFIMIVGGLIGLRIIFAVLSIV

SEQ ID NO: 42 - protein, 68 aa, Artificial Sequence
  Description of Artificial Sequence: Synthetic polypeptide
YTSEIYTLIEESQNQQEKNEQELLELDKWASLWNWFDITKWLWYIKIFIMIVGGLVGLRLVFTVLSIV

SEQ ID NO: 43 - protein, 71 aa, Artificial Sequence
  Description of Artificial Sequence: Synthetic polypeptide
SNLLRAIEAQQHLLQLTVWGIKQLQARVLAVERYLKDQFDITNWLWYIKIFIMIVGGLIGLRIVFAVLSIV

SEQ ID NO: 44 - protein, 70 aa, Artificial Sequence
  Description of Artificial Sequence: Synthetic polypeptide
SNLLRAIEAQQHLLKLTVWGIKQLQARVLAVERYLKDQFDISNWLWYIKIFIMIVGGLIGLRIVFAVLSV

SEQ ID NO: 45 - protein, 72 aa, Artificial Sequence
  Description of Artificial Sequence: Synthetic polypeptide
NNLLRAIEAQQHLLQLTVWGIKQLQARVLAVERYLKDQQFDITNWLWYIKIFIMIVGGLVGLRIVFAVLSIV

SEQ ID NO: 46 - protein, 71 aa, Artificial Sequence
  Description of Artificial Sequence: Synthetic polypeptide
SNLLRAIEAQQHMLQLTVWGIKQLQTRVLAIERYLKDQFDITNWLWYIKIFIMIVGGLIGLRIIFAVLSIV

SEQ ID NO: 47 - protein, 71 aa, Artificial Sequence
  Description of Artificial Sequence: Synthetic polypeptide
NNLLRAIEAQQRMLQLTVWGIKQLQARVLAVERYLGDQFDITKWLWYIKIFIMIVGGLVGLRLVFTVLSIV

SEQ ID NO: 48 - protein, 135 aa, Artificial Sequence
  Description of Artificial Sequence: Synthetic polypeptide
TLTVQARQLLSGIVQQQSNLLRAIEAQQHLLQLTVWGIKQLQARVLAVERYLKDQQLLGIWGCSGKLICTTTVPWNSSWSNKSQDEIWDNMTWMEWEREINNFDITNWLWYIKIFIMIVGGLIGLRIVFAVLSIV

SEQ ID NO: 49 - protein, 134 aa, Artificial Sequence
  Description of Artificial Sequence: Synthetic polypeptide
TLTVQARQLLSGIVQQQSNLLRAIEAQQHLLKLTVWGIKQLQARVLAVERYLKDQQLLGIWGCSGKLICTTNVPWNSSWSNKSQNEIWDNMTWLQWDKEISNFDISNWLWYIKIFIMIVGGLIGLRIVFAVLSV

SEQ ID NO: 50 - protein, 135 aa, Artificial Sequence
  Description of Artificial Sequence: Synthetic polypeptide
TLTVQARQLLSGIVQQQNNLLRAIEAQQHLLQLTVWGIKQLQARVLAVERYLKDQQLLGIWGCSGKLICTTAVPWNASWSNKSLDEIWDNMTWMEWEREIDNFDITNWLWYIKIFIMIVGGLVGLRIVFAVLSIV

SEQ ID NO: 51 - protein, 135 aa, Artificial Sequence
  Description of Artificial Sequence: Synthetic polypeptide
TLTVQARQLLSGIVQQQSNLLRAIEAQQHMLQLTVWGIKQLQTRVLAIERYLKDQQLLGIWGCSGKLICTTAVPWNSSWSNKSQEDIWDNMTWMQWDREISNFDITNWLWYIKIFIMIVGGLIGLRIIFAVLSIV

SEQ ID NO: 52 - protein, 135 aa, Artificial Sequence
  Description of Artificial Sequence: Synthetic polypeptide
TLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDNFDITKWLWYIKIFIMIVGGLVGLRLVFTVLSIV

SEQ ID NO: 53 - protein, 162 aa, Artificial Sequence
  Description of Artificial Sequence: Synthetic polypeptide
RAVGIGAVFLGFLGAAGSTMGAASITLTVQARQLLSGIVQQQSNLLRAIEAQQHLLQLTVWGIKQLQARVLAVERYLKDQQLLGIWGCSGKLICTTTVPWNSSWSNKSQDEIWDNMTWMEWEREINNYTDIIYSLIEESQNQQEKNEQELLALDKWASLWNW

SEQ ID NO: 54 - protein, 162 aa, Artificial Sequence
  Description of Artificial Sequence: Synthetic polypeptide
RAVGIGAVFLGFLGAAGSTMGAASITLTVQARQLLSGIVQQQSNLLRAIEAQQHLLKLTVWGIKQLQARVLAVERYLKDQQLLGIWGCSGKLICTTNVPWNSSWSNKSQNEIWDNMTWLQWDKEISNYTHIIYNLIEESQNQQEKNEQDLLALDKWANLWNW

SEQ ID NO: 55 - protein, 162 aa, Artificial Sequence
  Description of Artificial Sequence: Synthetic polypeptide
RAVGIGAMFLGFLGAAGSTMGAASMTLTVQARQLLSGIVQQQNNLLRAIEAQQHLLQLTVWGIKQLQARVLAVERYLKDQQLLGIWGCSGKLICTTAVPWNASWSNKSLDEIWDNMTWMEWEREIDNYTSLIYTLIEESQNQQEKNEQELLELDKWASLWNW

SEQ ID NO: 56 - protein, 161 aa, Artificial Sequence
  Description of Artificial Sequence: Synthetic polypeptide
AVGIGAVFLGFLGAAGSTMGAASITLTVQARQLLSGIVQQQSNLLRAIEAQQHMLQLTVWGIKQLQTRVLAIERYLKDQQLLGIWGCSGKLICTTAVPWNSSWSNKSQEDIWDNMTWMQWDREISNYTDTIYRLLEDSQNQQEKNEKDLLALDSWKNLWNW

SEQ ID NO: 57 - protein, 162 aa, Artificial Sequence
  Description of Artificial Sequence: Synthetic polypeptide
RAVGIGAVFLGFLGAAGSTMGAASMTLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDNYTSEIYTLIEESQNQQEKNEQELLELDKWASLWNW

SEQ ID NO: 58 - protein, 137 aa, Artificial Sequence
  Description of Artificial Sequence: Synthetic polypeptide
TLTVQARQLLSGIVQQQSNLLRAIEAQQHLLQLTVWGIKQLQARVLAVERYLKDQQLLGIWGCSGKLICTTTVPWNSSWSNKSQDEIWDNMTWMEWEREINNYTDIIYSLIEESQNQQEKNEQELLALDKWASLWNW

SEQ ID NO: 59 - protein, 137 aa, Artificial Sequence
  Description of Artificial Sequence: Synthetic polypeptide
TLTVQARQLLSGIVQQQSNLLRAIEAQQHLLKLTVWGIKQLQARVLAVERYLKDQQLLGIWGCSGKLICTTNVPWNSSWSNKSQNEIWDNMTWLQWDKEISNYTHIIYNLIEESQNQQEKNEQDLLALDKWANLWNW

SEQ ID NO: 60 - protein, 137 aa, Artificial Sequence
  Description of Artificial Sequence: Synthetic polypeptide
TLTVQARQLLSGIVQQQNNLLRAIEAQQHLLQLTVWGIKQLQARVLAVERYLKDQQLLGIWGCSGKLICTTAVPWNASWSNKSLDEIWDNMTWMEWEREIDNYTSLIYTLIEESQNQQEKNEQELLELDKWASLWNW

SEQ ID NO: 61 - protein, 137 aa, Artificial Sequence
  Description of Artificial Sequence: Synthetic polypeptide
TLTVQARQLLSGIVQQQSNLLRAIEAQQHMLQLTVWGIKQLQTRVLAIERYLKDQQLLGIWGCSGKLICTTAVPWNSSWSNKSQEDIWDNMTWMQWDREISNYTDTIYRLLEDSQNQQEKNEKDLLALDSWKNLWNW

SEQ ID NO: 62 - protein, 137 aa, Artificial Sequence
  Description of Artificial Sequence: Synthetic polypeptide
TLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDNYTSEIYTLIEESQNQQEKNEQELLELDKWASLWNW

SEQ ID NO: 63 - protein, 99 aa, Artificial Sequence
  Description of Artificial Sequence: Synthetic polypeptide
TLTVQARQLLSGIVQQQQLLGIWGCSGKLICTTTVPWNSSWSNKSQDEIWDNMTWMEWEREINNYTDIIYSLIEESQNQQEKNEQELLALDKWASLWNW

SEQ ID NO: 64 - protein, 99 aa, Artificial Sequence
  Description of Artificial Sequence: Synthetic polypeptide
TLTVQARQLLSGIVQQQQLLGIWGCSGKLICTTNVPWNSSWSNKSQNEIWDNMTWLQWDKEISNYTHIIYNLIEESQNQQEKNEQDLLALDKWANLWNW

SEQ ID NO: 65 - protein, 99 aa, Artificial Sequence
  Description of Artificial Sequence: Synthetic polypeptide
TLTVQARQLLSGIVQQQQLLGIWGCSGKLICTTAVPWNASWSNKSLDEIWDNMTWMEWEREIDNYTSLIYTLIEESQNQQEKNEQELLELDKWASLWNW

SEQ ID NO: 66 - protein, 99 aa, Artificial Sequence
  Description of Artificial Sequence: Synthetic polypeptide
TLTVQARQLLSGIVQQQQLLGIWGCSGKLICTTAVPWNSSWSNKSQEDIWDNMTWMQWDREISNYTDTIYRLLEDSQNQQEKNEKDLLALDSWKNLWNW

SEQ ID NO: 67 - protein, 99 aa, Artificial Sequence
  Description of Artificial Sequence: Synthetic polypeptide
TLTVQARLLLSGIVQQQQLLGIWGCSGKLICTTAVPWNASWSNKSLDRIWNNMTWMEWEREIDNYTSEIYTLIEESQNQQEKNEQELLELDKWASLWNW

SEQ ID NO: 68 - protein, 35 aa, Artificial Sequence
  Description of Artificial Sequence: Synthetic polypeptide
YTDIIYSLIEESQNQQEKNEQELLALDKWASLWNW

SEQ ID NO: 69 - protein, 35 aa, Artificial Sequence
  Description of Artificial Sequence: Synthetic polypeptide
YTHIIYNLIEESQNQQEKNEQDLLALDKWANLWNW

SEQ ID NO: 70 - protein, 35 aa, Artificial Sequence
  Description of Artificial Sequence: Synthetic polypeptide
YTSLIYTLIEESQNQQEKNEQELLELDKWASLWNW

SEQ ID NO: 71 - protein, 35 aa, Artificial Sequence
  Description of Artificial Sequence: Synthetic polypeptide
YTDTIYRLLEDSQNQQEKNEKDLLALDSWKNLWNW

SEQ ID NO: 72 - protein, 35 aa, Artificial Sequence
  Description of Artificial Sequence: Synthetic polypeptide
YTSEIYTLIEESQNQQEKNEQELLELDKWASLWNW

SEQ ID NO: 73 - protein, 38 aa, Artificial Sequence
  Description of Artificial Sequence: Synthetic polypeptide
SNLLRAIEAQQHLLQLTVWGIKQLQARVLAVERYLKDQ

SEQ ID NO: 74 - protein, 38 aa, Artificial Sequence
  Description of Artificial Sequence: Synthetic polypeptide
SNLLRAIEAQQHLLKLTVWGIKQLQARVLAVERYLKDQ

SEQ ID NO: 75 - protein, 38 aa, Artificial Sequence
  Description of Artificial Sequence: Synthetic polypeptide
NNLLRAIEAQQHLLQLTVWGIKQLQARVLAVERYLKDQ

SEQ ID NO: 76 - protein, 38 aa, Artificial Sequence
  Description of Artificial Sequence: Synthetic polypeptide
SNLLRAIEAQQHMLQLTVWGIKQLQTRVLAIERYLKDQ

SEQ ID NO: 77 - protein, 38 aa, Artificial Sequence
  Description of Artificial Sequence: Synthetic polypeptide
NNLLRAIEAQQRMLQLTVWGIKQLQARVLAVERYLGDQ

SEQ ID NO: 78 - protein, 137 aa, Artificial Sequence; identical to SEQ ID NO: 58
  Description of Artificial Sequence: Synthetic polypeptide

SEQ ID NO: 79 - protein, 137 aa, Artificial Sequence; identical to SEQ ID NO: 59
  Description of Artificial Sequence: Synthetic polypeptide

SEQ ID NO: 80 - protein, 137 aa, Artificial Sequence; identical to SEQ ID NO: 60
  Description of Artificial Sequence: Synthetic polypeptide

SEQ ID NO: 81 - protein, 137 aa, Artificial Sequence; identical to SEQ ID NO: 61
  Description of Artificial Sequence: Synthetic polypeptide

SEQ ID NO: 82 - protein, 137 aa, Artificial Sequence; identical to SEQ ID NO: 62
  Description of Artificial Sequence: Synthetic polypeptide

SEQ ID NO: 83 - protein, 102 aa, Artificial Sequence
  Description of Artificial Sequence: Synthetic polypeptide
TLTVQARQLLSGIVQQQSNLLRAIEAQQHLLQLTVWGIKQLQARVLAVERYLKDQQLLGIWGCSGKLICTTTVPWNSSWSNKSQDEIWDNMTWMEWEREINN

SEQ ID NO: 84 - protein, 102 aa, Artificial Sequence
  Description of Artificial Sequence: Synthetic polypeptide
TLTVQARQLLSGIVQQQSNLLRAIEAQQHLLKLTVWGIKQLQARVLAVERYLKDQQLLGIWGCSGKLICTTNVPWNSSWSNKSQNEIWDNMTWLQWDKEISN

SEQ ID NO: 85 - protein, 102 aa, Artificial Sequence
  Description of Artificial Sequence: Synthetic polypeptide
TLTVQARQLLSGIVQQQNNLLRAIEAQQHLLQLTVWGIKQLQARVLAVERYLKDQQLLGIWGCSGKLICTTAVPWNASWSNKSLDEIWDNMTWMEWEREIDN

SEQ ID NO: 86 - protein, 102 aa, Artificial Sequence
  Description of Artificial Sequence: Synthetic polypeptide
TLTVQARQLLSGIVQQQSNLLRAIEAQQHMLQLTVWGIKQLQTRVLAIERYLKDQQLLGIWGCSGKLICTTAVPWNSSWSNKSQEDIWDNMTWMQWDREISN

SEQ ID NO: 87 - protein, 102 aa, Artificial Sequence
  Description of Artificial Sequence: Synthetic polypeptide
TLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDN

SEQ ID NO: 88 - protein, 847 aa, Artificial Sequence
  Description of Artificial Sequence: Synthetic polypeptide
MRVKGIRKSYQYLWKGGTLLLGILMICSAVEKLWVTVYYGVPVWKEATTTLFCASDAKAYDTEVHNVWATHACVPTDPNPQEVVLENVTEHFNMWKNNMVEQMQEDIISLWDQSLKPCVKLTPLCVTLNCKDVNATNTTNDSEGTMERGEIKNCSFNITTSIRDEVQKEYALFYKLDVVPIDNNNTSYRLISCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGRAFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEVGKAMYAPPIRGQIRCSSNITGLLLTRDGGINENGTEIFRPGGGDMRDNWRSELYKYKVVKIEPLGVAPTKAKRRVVQREKRAVGIGAVFLGFLGAAGSTMGAASMTLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDNYTSEIYTLIEESQNQQEKNEQELLELDKWASLWNWFDITKWLWYIKIFIMIVGGLVGLRLVFTVLSIVNRVRQGYSPLSFQTLLPAPRGPDRPEGIEEEGGERDRDRSGRLVNGFLALIWVDLRSLCLFSYHRLRDLLLTVTRIVELLGRRGWEVLKYWWNLLQYWSQELKNSAVSLLNATAIAVAEGTDRIIEALQRTYRAILHIPTRIRQGLERALL

SEQ ID NO: 89 - protein, 829 aa, Artificial Sequence
  Description of Artificial Sequence: Synthetic polypeptide
MRVMGIQRNCQHLWRWGILIFGMLIICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTDVNATNNTTNNEEIKNCSFNITTEIRDKKKKVYALFYKLDVVPIDDNNSYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISRTKWNKTLQQVAKKLREHFNKTIIFNPSSGGDLEITTHSFNCGGEFFYCNTSELFNSTWNGTNNTITLPCRIKQIINMWQGVGQAMYAPPIEGKIRCTSNITGLLLTRDGGNNNTETFRPGGGDMRDNWRSELYKYKVVKIEPLGVAPTKAKRRVVEREKRAVGIGAVFLGFLGAAGSTMGAASITLTVQARQLLSGIVQQQSNLLRAIEAQQHLLQLTVWGIKQLQARVLAVERYLKDQQLLGIWGCSGKLICTTNVPWNSSWSNKSQDEIWDNMTWMEWDKEINNYTDIIYSLIEESQNQQEKNEQELLALDKWASLWNWFDITNWLWYIKIFIMIVGGLIGLRIVFAVLSIVNRVRQGYSPLSFQTLIPNPRGPDRPEGIEEEGGEQDRDRSIRLVNGFLALAWDDLRSLCLFSYHRLRDLILIAARTVELLGRRGWEALKYLWNLLQYWGQELKNSAISLLDTTAIAVAEGTDRVIEVVQRVCRAILNIPRRIRQGFERALL

SEQ ID NO: 90 - protein, 844 aa, Artificial Sequence
  Description of Artificial Sequence: Synthetic polypeptide
MRVMGIQRNCQHLLRWGTMILGMIIICSAAENLWVTVYYGVPVWKDAETTLFCASDAKAYETEMHNVWATHACVPTDPNPQEIHLENVTEEFNMWKNNMVEQMHTDIISLWDQSLKPCVKLTPLCVTLNCSNVNVTNNTTNTHEEEIKNCSFNMTTELRDKKQKVYSLFYRLDVVQINENNSNSSYRLINCNTSAITQACPKVSFEPIPIHYCAPAGFAILKCKDKEFNGTGPCKNVSTVQCTHGIKPVVSTQLLLNGSLAEEEVIIRSENITNNAKTIIVQLTKPVKINCTRPNNNTRKSIRIGPGQAFYATGDIIGDIRQAHCNVSRSEWNKTLQKVAKQLRKYFKNKTIIFTNSSGGDLEITTHSFNCGGEFFYCNTSGLFNSTWNNGTMKNTITLPCRIKQIINMWQRAGQAMYAPPIQGVIRCESNITGLLLTRDGGNNNTNETFRPGGGDMRDNWRSELYKYKVVKIEPLGVAPTRAKRRVVEREKRAVGIGAVFLGFLGAAGSTMGAASITLTVQARQLLSGIVQQQSNLLRAIEAQQHLLKLTVWGIKQLQARVLAVERYLKDQQLLGIWGCSGKLICTTNVPWNSSWSNKSQNEIWDNMTWLQWDKEISNYTHIIYNLIEESQNQQEKNEQDLLALDKWANLWNWFDISNWLWYIKIFIMIVGGLIGLRIVFAVLSVINRVRQGYSPLSFQTHTPNPRGLDRPGRIEEEGGEQGRDRSIRLVSGFLALAWDDLRSLCLFSYHRLRDFILIAARTVELLGHSSLKGLRLGWEGLKYLWNLLLYWGRELKISAINLVDTIAIAVAGWTDRVIEIGQRIGRAILHIPRRIRQGLERAL

SEQ ID NO: 91 - protein, 841 aa, Artificial Sequence
  Description of Artificial Sequence: Synthetic polypeptide
MRVKGIRKNYQHLWRWGTMLLGMLMICSAAEKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLMNATNTNTTIIYRWRGEIKNCSFNITTSIRDKVQKEYALFYKLDVVPIDNDNTSYRLISCNTSVITQACPKVSFEPIPIHYCAPAGFAILKCNDKKFNGTGPCTNVSTVQCTHGIRPVVSTQLLLNGSLAEEEVVIRSENFTDNAKTIIVQLNESVEINCTRPNNNTRKSIHIGPGRAFYTTGEIIGDIRQAHCNISRAKWNNTLKQIVKKLREQFGNKTIVFNQSSGGDPEIVMHSFNCGGEFFYCNTTQLFNSTWNGTWNNTEGNITLPCRIKQIINMWQEVGKAMYAPPIRGQIRCSSNITGLLLTRDGGNNETEIFRPGGGDMRDNWRSELYKYKVVKIEPLGVAPTKAKRRVVQREKRAVGIGAMFLGFLGAAGSTMGAASMTLTVQARQLLSGIVQQQNNLLRAIEAQQHLLQLTVWGIKQLQARVLAVERYLKDQQLLGIWGCSGKLICTTAVPWNASWSNKSLDEIWDNMTWMEWEREIDNYTSLIYTLIEESQNQQEKNEQELLELDKWASLWNWFDITNWLWYIKIFIMIVGGLVGLRIVFAVLSIVNRVRQGYSPLSFQTRLPAPRGPDRPEGIEEEGGERDRDRSGRLVDGFLALIWDDLRSLCLFSYHRLRDLLLIVTRIVELLGRRGWEVLKYWWNLLQYWSQELKNSAVSLLNATAIAVAEGTDRVIEVVQRACRAILHIPRRIRQGLERALL

SEQ ID NO: 92 - protein, 835 aa, Artificial Sequence
  Description of Artificial Sequence: Synthetic polypeptide
MRVRGILRNCQQWWIWGILGFWMLMICNVVGNLWVTVYYGVPVWKEAKTTLFCASDAKAYEKEVHNVWATHACVPTDPNPQEIVLENVTENFNMWKNDMVDQMHEDIISLWDQSLKPCVKLTPLCVTLNCTNATNATNTMGEIKNCSFNITTELRDKKQKVYALFYRLDIVPLNENNSYRLINCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNESVEIVCTRPNNNTRKSIRIGPGQTFYATGDIIGDIRQAHCNISEDKWNKTLQKVSKKLKEHFPNKTIKFEPSSGGDLEITTHSFNCRGEFFYCNTSKLFNSTYNSTNSTITLPCRIKQIINMWQEVGRAMYAPPIAGNITCKSNITGLLLTRDGGKNNTETFRPGGGDMRDNWRSELYKYKVVEIKPLGIAPTKAKRRVVEREKRAVGIGAVFLGFLGAAGSTMGAASITLTVQARQLLSGIVQQQSNLLRAIEAQQHMLQLTVWGIKQLQTRVLAIERYLKDQQLLGIWGCSGKLICTTAVPWNSSWSNKSQEDIWDNMTWMQWDREISNYTDTIYRLLEDSQNQQEKNEKDLLALDSWKNLWNWFDITNWLWYIKIFIMIVGGLIGLRIIFAVLSIVNRVRQGYSPLSFQTLTPNPRGPDRLGRIEEEGGEQDRDRSIRLVSGFLALAWDDLRSLCLFSYHRLRDFILIAARAVELLGRSSLRGLQRGWEALKYLGSLVQYWGLELKKSAISLLDTIAIAVAEGTDRIIELIQRICRAIRNIPRRIRQGFEAALQ

SEQ ID NO: 93 - protein, 847 aa, Artificial Sequence; identical to SEQ ID NO: 88
  Description of Artificial Sequence: Synthetic polypeptide

SEQ ID NO: 94 - protein, 863 aa, Artificial Sequence
  Description of Artificial Sequence: Synthetic polypeptide
MGAMGIQMNWQNLWRWGTMILGMLIICSVAEKSWVTVYYGVPVWRDAETTLFCASDAKAHDKEVHNVWATHACVPTDPNPQEMILENVTEDFNMWKNSMVEQMHTDIISLWDQSLKPCVKLTPLCVTLNCSDSNITSNSTSNSTKDSATLDMKSEIQNCSFNMTTELRDKKQKVYSLFYRLDVVQINENSSDYRLINCNTSAITQACPKVTFEPIPIHYCAPAGFAILKCNDKKFNGTGPCTNVSTVQCTHGIKPVVTTQLLLNGSLAEEEVMIRSENITENAKNIIVQFKEPVQIICIRPGNNTRKSVHIGPGQAFYATGDIIGDIRQAHCNVSRELWNKTLQEVATQLRKHFRNNTKIIFTNSSGGDVEITTHSFNCGGEFFYCDTSGLFNSSWTASNDSMQEAHSTESNITLQCRIKQIINMWQRAGQAMYAPPIPGIIRCESNITGLILTRDGGEGNNSTNETFRPVGGNMRDNWRSELYKYKVVKVEPLGVAPTKSRRRVVEREKRAVGLGAVFIGFLGAAGSTMGAASMTLTVQARQLLSGIVQQQSNLLRAIEAQQHLLKLTVWGIKQLQARVLAVERYLRDQQLLGIWGCSGKLICTTNVPWNSSWSNKSLDEIWENMTWMQWDKEVSNYTQMIYNLLEESQNQQEKNEQELLALDKWANLWNWFNISNWLWYIKIFIMIVGGLIGLRIVFAVLSVINRVRQGYSPLSFQTHTPNPRGLDRPGRIEEEGGEQDRDRSIRLVSGFLALAWDDLRSLCLFSYHRLRDFILIAARTLELLGHNSLKGLRLGWEGLKYLWNLLAYWGRELKISAISLVDSIAIAVAGWTDRIIEIVQAIGRAILHIPRRIRQGLERALI

SEQ ID NO: 95 - protein, 861 aa, Artificial Sequence
  Description of Artificial Sequence: Synthetic polypeptide
MRVKEIRRNCQRLRRWGTMLLGMLMICSATEQLWVTVYYGVPVWKEATTTLFCASDAKAYVTEKHNVWATHACVPTDPNPQEVVLENVTENFNMWKNNMVEQMHEDIISLWEQSLKPCVKLTPLCVTLNCTDKLRNDTSGTNSSSWEKVQKGEIKNCSFNITTGIRGRVQEYSLFYKLDVIPIDSRNNSNNSTEFSSYRLISCNTSVITQACPKISFEPIPIHYCAPAGFAILKCNDKKFNGTGPCKNVSTVQCTHGIKPVVSTQLLLNGSLAEEEVVIRSENFTNNVKSIIVQLNKSVVINCTRPNNNTRKSIHIGAGKALYTGEIIGDIRQAHCNLSRAQWNNTLKQIVIKLREQFGNKTIVFNQSSGGDVEIVMHSFNCGGEFFYCNSTQLFNSTWNGNDTWNDTWKDTTNDNITLPCRIKQIVNMWQKVGKAMYAPPIRGQIRCSSKITGLILTRDGGTNGTNETETFRPGGGNMKDNWRSELYKYKVVKIEPLGIAPTKAKRRVVQREKRAVGTIGAMFLGFLGAAGSTMGAASLTLTVQARLLLSGIVQQQNNLLRAIEAQQHLLQLTVWGIKQLQARVLAVERYLRDQQLLGIWGCSGRLICTTNVPWNTSWSNRSLNYIWDNMTWMQWDREINNYTDYIYTLLEDAQNQQEKNEQELLELDKWASLWNWFDITNWLWYIKIFIMIVGGLIGLRIVFAVLSIVNRVRQGYSPLSLQTHLPARRGPDRPEGIGEEGGERDRDRSVRLVHGFLALVWEDLRSLCLFSYHRLRDLLLIVARTVEILGQRGWEALKYWWNLLLYWSLELKNSAVSLVDTIAIAVAEGTDRIIEIARRIFRAFLHIPTRIRQGLERALL

SEQ ID NO: 96 - protein, 854 aa, Artificial Sequence
  Description of Artificial Sequence: Synthetic polypeptide
MRVKGIQRNWPQWWIWGILGFWMIIICRVVGNLWVTVYYGVPVWTEAKTTLFCASDAKAYEREVHNVWATHACVPTDPNPQEIVLGNVTENFNMWKNDMVDQMHEDIISIWDQSLKPCVKLTPLCVTLNCTDVKVNATSNGTTTYNNSIDSMNGEIKNCSFNITTEIRDKKQKVYALFYRPDVVPLNENSSSYILINCNTSTTTQACPKVSFDPIPIHYCAPAGYAILKCNNKTFNGTGPCHNVSTVQCTHGIKPVVSTQLLLNGSLAEEEIIIRSENLTNNAKTIIVHLNESIEIVCTRPNNNTRKSIRIGPGQTVYATNDIIGDIRQAHCNISKTKWNTTLEKVKEKLKEHFPSKAITFQPHSGGDLEVTTHSFNCRGEFFYCDTTKLFNESNLNTTNTTTLTLPCRIKQIVNMWQGVGRAMYAPPVEGNITCNSSITGLLLVRDGGNTSNSTPEIFRPGGGNMKDNWRSELYKYKVVEIKPLGVAPTKAKRRVVEREKRAVGIGAVLFGFLGAAGSTMGAASITLTVQARQLLSGIVQQQSNLLRAIEAQQHMLQLTVWGIKQLQARVLAIERYLKDQQLLGLWGCSGKLICPTTVPWNSSWSNKSQTDIWDNMTWMQWDREISNYTGTIYKLLEESQNQQEKNEKDLLALDSWKNLWSWFDITNWLWYIKIFIMIVGGLIGLRIIFGVLSIVKRVRQGYSPLSFQTLTPNPRGLDRLGRIEEEGGEQDKDRSIRLVNGFLALAWDDLRSLCLFSYHRLRDFILVAARAVELLGRSSLRGLQRGWEALKYLGNLVQYGGLELKRRAISLFDTIAIAVAEGTDRILEVILRIIRAIRNIPTRIRQGFEAALL

SEQ ID NO: 97 - protein, 854 aa, Artificial Sequence
  Description of Artificial Sequence: Synthetic polypeptide
MRVKETQMNWPNLWKWGTLILGLVIICSASDNLWVTVYYGVPVWRDADTTLFCASDAKAHETEVHNVWATHACVPTDPNPQEIHLENVTENFNMWRNNMVEQMQEDVISLWDQSLKPCVKLTPLCVTLNCTNANWTNSNNTTNGPNKIGNITDEVKNCTFNMTTELKDKKQKVHALFYKLDIVQINSSEYRLINCNTSVIKQACPKISFDPIPIHYCTPAGYAILKCNDKNFNGTGPCKNVSSVQCTHGIKPVVSTQLLLNGSLAEEEIIIRSENLTNNAKTIIVHLNKSVEINCTRPSNNTRTSITMGPGQVFYRTGDIIGDIRKAYCEINGIKWNEVLVQVTGKLKEHFNKTIIFQPPSGGDLEIITHHFSCRGEFFYCNTTKLFNNTCIGNTSMEGCNNTIILPCKIKQIINMWQGVGQAMYAPPISGRINCVSNITGILLTRDGGADNNTTNETFRPGGGNIKDNWRSELYKYKVVEIEPLGIAPTRAKRRVVEREKRAVGIGAMIFGFLGAAGSTMGAASITLTVQARQLLSGIVQQQSNLLRAIEAQQHLLQLTVWGIKQLQARVLAVERYLKDQKFLGLWGCSGKIICTTAVPWNSSWSNKSFEEIWDNMTWIEWEREISNYTSQIYEILTESQNQQDRNEKDLLELDKWASLWNWFDITNWLWYIKIFIIIVGSLIGLRIIFAVLSIVNRVRQGYSPLSFQTPTHHQREPDRPEEIGEGGGEQSKDRSVRLVSGFLALAWDDLRSLCLFSYHLLRDFILIAARTVELLGHSSLKGLRRGWEGLKYLGNLLLYWGQEIKISAISLLNATAIAVAGWTDRVIEVAQRAWRALLHIPRRIRQGLERALL

SEQ ID NO: 98 - protein, 854 aa, Artificial Sequence
  Description of Artificial Sequence: Synthetic polypeptide
MRVKGIQRNWQHLWNWGILILGLVIICSAEKLWVTVYYGVPVWEDANAPLFCASDAKAHSTESHNIWATHACVPTDPSPQEINMRNVTENFNMWKNNMVEQMHEDIISLWDESLKPCVKLTPLCVTLNCTEINNNSTRNITEEYRMTNCSFNMTTELRDKKKAEYALFYRTDVVPINEMNNENNGTNSTWYRLTNCNVSTIKQACPKVTFEPIPIHYCAPAGFAILKCVDKKFNGTGTCNNVSTVQCTHGIKPVVSTQLLLNGSLAEKDIIISSENISDNAKVIIVHLNRSVEINCTRPNNNTRRSVAIGPGQAFYTTGEVIGDIRKAHCNVSWTKWNETLRDVQAKLQEYFINKSIEFNSSSGGDLEITTHSFNCGGEFFYCNTSGLFNNSILKSNISENNDTITLNCKIKQIVRMWQRVGQAMYAPPIAGNITCRSNITGLILTRDGGDNNSTSEIFRPGGGDMKNNWRSELYKYKTVKIKSLGIAPTRARRRVVEREKRAVGVGAIFLGFLGTAGSTMGAASITLTVQVRQLLSGIVQQQSNLLRAIEAQQHLLQLTVWGIKQLRARVLALERYLKDQQLLGIWGCSGKLICTTNVPWNTSWSNKSYNEIWENMTWIEWEREIDNYTYHIYSLIEQSQIQQEKNEQDLLALDQWASLWSWFSISNWLWYIRIFVMIVGGLIGLRIVFAVLSIVNRVRQGYSPLSFQTLLHHQREPDRPAGIEEGGGEQDRDRSIRLVSGFLALAWDDLRSLCLFSYHRLRDFILIAARTVELLGRNSLKGLRLGWEALKYLWNLLLYWARELKNSAINLLDTIAIAVANWTDRVIEVAQRAGRAVLNIPRRIRQGLERALL